(19) United States
(12) Patent Application Publication
(10) Pub. No.: US 2010/0004301 A1
(43) Pub. Date: Jan. 7, 2010

(54) Benzoxazoles Useful in the Treatment of Inflammation

(76) Inventors: Benjamin Pelcman, Solna (SE); Kristofer Olofsson, Solna (SE); Ivars Kalvins, Riga (LV); Edgars Suna, Riga (LV); Vita Ozola, Riga (LV); Wesley Schaal, Solna (SE)

Correspondence Address: MORGAN LEWIS & BOCKIUS LLP, 1111 PENNSYLVANIA AVENUE NW, WASHINGTON, DC, 20004, US

(21) Appl. No.: 12/518,370

(22) PCT Filed: Dec. 12, 2007
(86) PCT No.: PCT/GB2007/004747

§ 371 Date: Sep. 17, 2009

Related U.S. Application Data
(60) Provisional application No. 60/874,684, filed on Dec. 14, 2006.

(51) Int. Cl. A61K 31/423; C07D 263/56

(52) U.S. Cl. 514/375; 548/224

(57) ABSTRACT

There is provided the use of a compound of formula I,

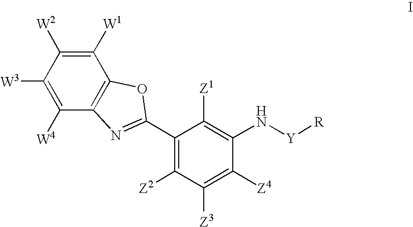

wherein Y, W¹ to W⁴, Z¹ to Z⁴ and R have meanings given in the description, and pharmaceutically-acceptable salts thereof, for the manufacture of a medicament for the treatment of a disease in which inhibition of the activity of a member of the MAPEG family is desired and/or required, and particularly in the treatment of inflammation.

### FIELD OF THE INVENTION

This invention relates to a novel pharmaceutical use of certain compounds, some of which compounds are not known as pharmaceuticals. In particular, this invention relates to the use of such compounds as inhibitors of enzymes belonging to the membrane-associated proteins in the eicosanoid and glutathione metabolism (MAPEG) family. Members of the MAPEG family include the microsomal prostaglandin E synthase-1 (mPGES-1), 5-lipoxygenase-activating protein (FLAP), leukotriene C₄ synthase and microsomal glutathione S-transferases (MGST1, MGST2 and MGST3). Thus, the compounds are of potential utility in the treatment of inflammatory diseases including respiratory diseases.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Inflammatory diseases that affect the population include asthma, inflammatory bowel disease, rheumatoid arthritis, osteoarthritis, rhinitis, conjunctivitis and dermatitis.

Inflammation is also a common cause of pain. Inflammatory pain may arise for numerous reasons, such as infection, surgery or other trauma. Moreover, several diseases including malignancies and cardioavascular diseases are known to have inflammatory components adding to the symptomatology of the patients.

Asthma is a disease of the airways that contains elements of both inflammation and bronchoconstriction. Treatment regimens for asthma are based on the severity of the condition. Mild cases are either untreated or are only treated with inhaled β-agonists which affect the bronchoconstriction element, whereas patients with more severe asthma typically are treated regularly with inhaled corticosteroids which to a large extent are anti-inflammatory in their nature.

Another common disease of the airways with inflammatory and bronchoconstrictive components is chronic obstructive pulmonary disease (COPD). The disease is potentially lethal, and the morbidity and mortality from the condition is considerable. At present, there is no known pharmacological treatment capable of changing the course of the disease.

The cyclooxygenase (COX) enzyme exists in two forms, one that is constitutively expressed in many cells and tissues (COX-1), and one that in most cells and tissues is induced by pro-inflammatory stimuli, such as cytokines, during an inflammatory response (COX-2).

COXs metabolize arachidonic acid to the unstable intermediate prostaglandin H₂ (PGH₂). PGH₂ is further metabolized to other prostaglandins including PGE₂, PGF_{2α}, PGD₂, prostacyclin and thromboxane A₂. These arachidonic acid metabolites are known to have pronounced physiological and pathophysiological activity including pro-inflammatory effects.

PGE₂ in particular is known to be a strong pro-inflammatory mediator, and is also known to induce fever and pain. Consequently, numerous drugs have been developed with a view to inhibiting the formation of PGE₂, including “NSAIDs” (non-steroidal antiinflammatory drugs) and “coxibs” (selective COX-2 inhibitors). These drugs act predominantly by inhibition of COX-1 and/or COX-2, thereby reducing the formation of PGE₂.

However, the inhibition of COXs has the disadvantage that it results in the reduction of the formation of all metabolites downstream of PGH₂, some of which are known to have beneficial properties. In view of this, drugs which act by inhibition of COXs are therefore known/suspected to cause adverse biological effects. For example, the non-selective inhibition of COXs by NSAIDs may give rise to gastrointestinal side-effects and affect platelet and renal function. Even the selective inhibition of COX-2 by coxibs, whilst reducing such gastrointestinal side-effects, is believed to give rise to cardiovascular problems.

An alternative treatment of inflammatory diseases that does not give rise to the above-mentioned side effects would thus be of real benefit in the clinic. In particular, a drug that inhibits (preferably selectively) the transformation of PGH₂ to the pro-inflammatory mediator PGE₂ might be expected to reduce the inflammatory response in the absence of a corresponding reduction of the formation of other, beneficial arachidonic acid metabolites. Such inhibition would accordingly be expected to alleviate the undesirable side-effects mentioned above.

PGH₂ may be transformed to PGE₂ by prostaglandin E synthases (PGES). Two microsomal prostaglandin E synthases (mPGES-1 and mPGES-2), and one cytosolic prostaglandin E synthase (cPGES) have been described.

The leukotrienes (LTs) are formed from arachidonic acid by a set of enzymes distinct from those in the COX/PGES pathway. Leukotriene B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. The biological activities of the CysLTs are mediated through two receptors designated CysLT₁ and CysLT₂. As an alternative to steroids, leukotriene receptor antagonists (LTRas) have been developed in the treatment of asthma. These drugs may be given orally, but do not control inflammation satisfactorily. The presently used LTRas are highly selective for CysLT₁. It may be hypothesized that better control of asthma, and possibly also COPD, may be attained if the activity of both of the CysLT receptors could be reduced. This may be achieved by developing unselective LTRas, but also by inhibiting the activity of proteins, e.g. enzymes, involved in the synthesis of the CysLTs. Among these proteins, 5-lipoxygenase, 5-lipoxygenase-activating protein (FLAP), and leukotriene C₄ synthase may be mentioned. A FLAP inhibitor would also decrease the formation of the proinflammatory LTB₄.

mPGES-1, FLAP and leukotriene C₄ synthase belong to the membrane-associated proteins in the eicosanoid and glutathione metabolism (MAPEG) family. Other members of this family include the microsomal glutathione S-transferases (MGST1, MGST2 and MGST3). For a review, c.f. P.-J. Jacobsson et al in Am. J. Respir. Crit. Care Med. 161, S20 (2000). It is well known that compounds prepared as antagonists to one of the MAPEGs may also exhibit inhibitory activity towards other family members, cf. J. H Hutchinson et al in J. Med. Chem. 38, 4538 (1995) and D. Claveau et al in J. Immunol. 170, 4738 (2003). The former paper also describes that such compounds may also display notable cross-reactivity with proteins in the arachidonic acid cascade that do not belong to the MAPEG family, e.g. 5-lipoxygenase.

Thus, agents that are capable of inhibiting the action of mPGES-1, and thus reducing the formation of the specific arachidonic acid metabolite PGE₂, are likely to be of benefit in the treatment of inflammation. Further, agents that are capable of inhibiting the action of the proteins involved in the synthesis of the leukotrienes are also likely to be of benefit in the treatment of asthma and COPD.

The listing or discussion of an apparently prior-published document in this specification should not necessarily be taken as an acknowledgement that the document is part of the state of the art or is common general knowledge.

International patent application WO 03/037274 discloses various compounds for use as sodium channel blockers and thus in the treatment of inflammation. One compound disclosed is a benzoxazole which is substituted in the 2-position by a phenyl ring, which phenyl ring is further substituted in the meta position with a 1-chlorophenyl-5-trifluoromethyl-4-pyrazolylcarboxamido moiety.

International patent application WO 2007/042816 discloses various benzoxazoles, which constitute one aromatic group in a series of three. Such compounds are disclosed as being useful as inhibitors of a member of the MAPEG family, and thus in the treatment of inflammation. However, there is no mention or suggestion in this document of compounds in which any one of the three aromatic groups are themselves further substituted (via a linker group or otherwise) with another aromatic group.

US patent application U.S. Pat. No. 4,038,396 and journal article Journal of Medicinal Chemistry, 1978, Vol. 21, No. 11 by Clark et al both disclose various compounds for potential use in the treatment of inflammation, including compounds containing a series of three aromatic rings of which one may be an oxazolopyridine group. However, none of those aromatic rings may represent a benzoxazole group.

International patent applications WO 2005/030705, WO 2005/030704, WO 2004/032716, WO 03/045929, WO 03/045930, WO 03/037274 and WO 03/011219, and journal articles Chemistry and Biology (2004), 11 (9), 1293-1299 by Kao et al and Biochemistry and Medicinal Chemistry Letters (2004), 14 (6), 1455-1459 by Gong et al all disclose various benzoxazoles, or analogues thereof (e.g. oxazolopyridines) that are useful as pharmaceuticals. However none of these documents suggest the use of such compounds as inhibitors of a member of the MAPEG family, and thus in the treatment of inflammation.

International patent applications WO 2004/046122 and WO 2004/046123 disclose benzoxazole derivatives that may be useful as heparanase inhibitors, and thus in the treatment of inflammation. However, the former document does not mention or suggest compounds that are not substituted (via a linker group or otherwise) by a carboxy or tetrazolyl group. Further, the latter document does not mention or suggest benzoxazoles substituted with a phenyl ring, in which that phenyl ring is substituted by an aromatic amido group.

International patent application WO 2004/035522 discloses inter alia benzoxazoles for use as probes for the imaging diagnosis of diseases in which prion protein is accumulated. This document does not mention or suggest the use of the compounds disclosed therein as inhibitors of a member of the MAPEG family, and thus in the treatment of inflammation.

International patent application WO 96/11917 discloses heteroaryl groups including benzoxazoles that may be useful as PDE IV inhibitors, and therefore in the treatment of inflammation. However, there is no disclosure in this document of benzoxazoles that are substituted in the 2-position with two consecutive aromatic groups, nor is there the suggestion of the use of the compounds disclosed therein as inhibitors of a member of the MAPEG family.

International patent application WO 2004/089470 discloses various compounds that may be useful in modulating the activity of 11 β-hydroxysteroid dehydrogenase type 1, for use in, for example, cancer. International applications WO 2004/089416 and WO 2004/089415 also disclose the use of these compounds in combination therapy. However, none of these documents disclose or suggest the use of such compounds as inhibitors of a member of the MAPEG family.

U.S. Pat. No. 5,298,189 discloses various compounds that may comprise a series of three rings. However, such compounds are only disclosed as fluorescent compounds for use in the detection of high energy particles.

International patent application WO 2007/019417 discloses various compounds as sirtuin modulators. However, there is no specific disclosure in this document of compounds containing a series of three aromatic rings, one of which is a benzoxazole, and which series of three aromatic rings is further substituted with another aromatic ring.

Finally, several compounds have been disclosed in the Chemcats database, for instance in catalogues such as AKos, TOSlab, Synthetic and Natural Product List, ChemStar Product List and Interchim Intermediates. However, such compounds do not appear to have any use ascribed to them.

### DISCLOSURE OF THE INVENTION

A compound of formula I,

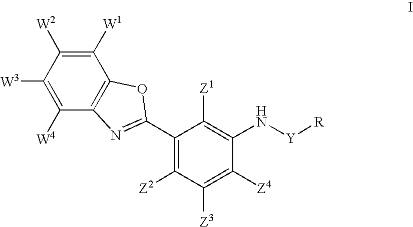

wherein

R represents aryl or heteroaryl, both of which are optionally substituted by one or more substituents selected from X¹;

Y represents —C(O)— or —S(O)₂—;

W¹ to W⁴ independently represent hydrogen or a substituent selected from X²;

Z¹ to Z⁴ independently represent hydrogen or a substituent selected from X³;

X¹, X² and X³ independently represent halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ;

R^{3b} to R^{3h}, R^{3j}, R^{3k}, R³ⁿ, R^{4a} to R⁴ⁱ, R^{5a}, R^{5b}, R^{5d} and R^{5f} to R⁵ⁱ independently represent H or R^{3a}; or

any of the pairs R^{4a} and R^{5a}, R^{4b} and R^{5b}, R^{4d} and R^{5d}, R^{4f} and R^{5f}, R^{4g} and R^{5g}, R^{4h} and R^{5h} or R⁴ⁱ and R⁵ⁱ may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{3a};

R³ⁱ, R^{3m} and R^{3p} independently represent R^{3a};

R^{3a} represents, on each occasion when mentioned above, aryl, heteroaryl (which latter two groups are optionally substituted by one or more substituents selected from G³) or C₁₋₆ alkyl optionally substituted by one or more substituents selected from aryl, heteroaryl (which latter two groups are optionally substituted by one or more substituents selected from G⁴), F, Cl, ═O, —OR^{6a} and —N(R^{6a})R^{7b};

R^{6a} and R^{6b} independently represent H, aryl, heteroaryl (which latter two groups are optionally substituted by one or more groups selected from G⁵) or C₁₋₆ alkyl optionally substituted by one or more substituents selected from aryl, heteroaryl (which latter two groups are optionally substituted by one or more groups selected from G⁶), F, Cl, ═O, —OR^{8a}, —N(R^{9a})R^{10a} and —S(O)₂-G¹;

R^{7b} represents H, —S(O)₂CH₃, —S(O)₂CF₃ or C₁₋₆ alkyl optionally substituted by one or more substituents selected from F, Cl, ═O, —OR^{11a}, —N(R^{12a})R^{13a} and —S(O)₂-G²; or R^{6b} and R^{7b} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or C₁₋₃ alkyl optionally substituted by one or more fluoro atoms;

G¹ and G² independently represent —N(R^{14a})R^{15a} or C₁₋₆ alkyl optionally substituted by one or more substituents selected from F, Cl, ═O, —OR^{16a} and —N(R^{17a})R^{18a};

R^{8a} and R^{11a} independently represent H, —CH₃, —CH₂CH₃ or —CF₃;

R^{9a}, R^{10a}, R^{12a}, R^{13a}, R^{14a}, R^{15a}, R^{16a}, R^{17a} and R^{18a} independently represent H, —CH₃ or —CH₂CH₃;

G³, G⁴, G⁵ and G⁶ independently represent halo, —R^{20a}, —CN, —C(O)R^{20b}, —C(O)OR^{20c}, —C(O)N(R^{21a})R^{22a}, —N(R^{21b})R^{22b}, —N(R^{20d})C(O)R^{21c}, —N(R^{20e})C(O)N(R^{21d})R^{22d}, N(R^{20f})C(O)OR^{21e}, —N₃, —NO₂, —N(R^{20g})S(O)₂N(R^{21f})R^{22f}, —OR^{20h}, —OC(O)N(R^{21g})R^{22g}, —OS(O)₂R²⁰ⁱ, —S(O)R^{20j}, —N(R^{20k})S(O)₂R^{20m}, —OC(O)R²⁰ⁿ, —OC(O)OR^{20p}, —S(O)₂N(R^{21h})R^{22h} or —OS(O)₂N(R²¹ⁱ)R²²ⁱ;

m represents 0, 1 or 2;

R^{20b} to R^{20h}, R^{20j}, R^{20k}, R²⁰ⁿ, R^{21a} to R²¹ⁱ, R^{22a}, R^{22b}, R^{22d} and R^{22f} to R²²ⁱ independently represent H or R^{20a}; or

any of the pairs R^{21a} and R^{22a}, R^{21b} and R^{22b}, R^{21d} and R^{22d}, R^{21f} and R^{22f}, R^{21g} and R^{22g}, R^{21h} and R^{22h} or R²¹ⁱ and R²²ⁱ may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{20a};

R²⁰ⁱ, R^{20m} and R^{20p} independently represent R^{20a};

R^{20a} represents, on each occasion when mentioned above, C₁₋₆ alkyl (optionally substituted by one or more substituents selected from ═O and T¹) or aryl or heteroaryl (which latter two groups are optionally substituted by one or more substituents selected from T²);

T¹ and T² independently represent F, Cl, —OR^{23a} or —N(R^{23b})R^{24b};

R^{23a}, R^{23b} and R^{24b} independently represent H, C₁₋₃ alkyl (optionally substituted by one or more substituents selected from ═O and T³) or aryl or heteroaryl (which latter two groups are optionally substituted by one or more substituents selected from T⁴);

T³ and T⁴ independently represent F, Cl, —OR^{25a} or —N(R^{25b})R^{26b};

R^{25a}, R^{25b} and R^{26b} independently represent H or C₁₋₃ alkyl optionally substituted by one or more fluoro atoms;

wherein:

at least one X¹, X² or X³ group is present and represents —R^{3a}, —C(O)R^{3b}, —C(O)OR^{3c}, —C(O)N(R^{4a})R^{5a}, N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ, in which the foregoing groups contain at least one (e.g. one) aryl or heteroaryl group (both of which are optionally substituted as defined above),

or a pharmaceutically acceptable salt thereof,

provided that:

when Y represents —C(O)—:

(a) W¹ to W⁴ and Z¹ to Z⁴ all represent H, then R does not represent 5-trifluoromethyl-N-(4-chlorophenyl)-pyrazol-4-yl;

(b) W¹ to W⁴, Z¹, Z² and Z⁴ all represent H, R represents unsubstituted phenyl and Z³ represents —N(R^{3d})C(O)R^{4c} in which R^{3d} represents H, then R^{4c} does not represent unsubstituted phenyl;

(c) W¹, W³, W⁴ and Z¹ to Z⁴ represent H, R represents unsubstituted phenyl, then W² does not represent 2-furanyl or 2-fluorophenyl;

(d) W¹ to W⁴, Z¹ and Z⁴ all represent H, Z² represents —OH and Z³ represents —C(O)R^{3b}, then R and R^{3b} do not both represent unsubstituted phenyl or 4-fluorophenyl;

(e) W¹, W⁴, Z¹ and Z³ all represent hydrogen:
(I) W² and Z² represent hydrogen, Z⁴ represents chloro, then when:
   (A) W³ represents —CH(CH₂CH₃)CH₃ (i.e. 1-methylpropyl), then R does not represent 4-(benzyloxy)-phenyl, 3-(benzyloxy)phenyl or 4-(phenyl)phenyl;
   (B) W³ represents isopropyl, then R does not represent 3-(benzyloxy)phenyl;
(II) W² and Z⁴ represent hydrogen, W³ represents ethyl:
   (A) Z² represents hydroxy, then R does not represent (4-phenyl)phenyl;
   (B) Z² represent hydrogen, then R does not represent 3-(2-oxo-2H-1-benzopyran-3-yl)-phenyl (i.e. 3-(2-oxo-2H-chromen-3-yl)-phenyl);
(III) W³ and Z² represent hydrogen, W² represents methyl, Z⁴ represents chloro, then R does not represent 3-(benzyloxy)phenyl;
(IV) W², W³, Z² and Z⁴ represent hydrogen, then R does not represent 3-(phenoxymethyl)phenyl or 2-(2,4-dimethylphenyl)-2,3-dihydro-1,3-dioxo-1H-isoindol-5-yl;

(f) W⁴ and Z³ represent hydrogen, R represents 2-furanyl substituted in the 5-position (only) by X¹, then:
(I) when W¹, W², Z¹ and Z² represent hydrogen:
   (A) Z⁴ represents hydrogen, W³ represents 1-methylpropyl, then X¹ does not represent 3-nitrophenyl;
   (B) Z⁴ represents hydrogen, W³ represents isopropyl, then X¹ does not represent 2,5-dichlorophenyl;
   (C) Z⁴ represents hydrogen, W³ represents chloro, then X¹ does not represent 2,3-dichlorophenyl;
   (D) Z⁴ represents methyl, W³ represents isopropyl, then X¹ does not represent 3-chloro-4-methylphenyl;
(II) when W¹ represents hydrogen, W² and W³ represent methyl:
   (A) Z¹ and Z² represent hydrogen, Z⁴ represents —OCH₃, then X¹ does not represent 2,5-dichlorophenyl;
   (B) Z¹ represents methyl, Z² and Z⁴ represent hydrogen, then X¹ does not represent 4-(carboethoxy)phenyl;
   (C) Z¹, Z² and Z⁴ represent hydrogen, then X¹ does not represent 2,5-dichlorophenyl;
(III) W¹, W², W³ and Z² represent hydrogen:
   (A) Z⁴ represents hydrogen, Z¹ represents methyl, then X¹ does not represent 3-chloro-4-methylphenyl or 4-bromophenyl;
   (B) Z¹ represents hydrogen, Z⁴ represents —OCH₃, then X¹ does not represent 3-chloro-2-methylphenyl;
(IV) X¹ does not represent 2-nitrophenyl when:
   (A) W¹, W², Z¹ and Z² represent hydrogen, W³ represents —OCH₃ and Z⁴ represents methyl;
   (B) W¹, W², W³, Z¹ and Z² represent hydrogen, and Z⁴ represents —OCH₃;
   (C) W¹, W², Z² and Z⁴ represent hydrogen, W³ represents chloro and Z¹ represents methyl;
   (D) W¹, Z² and Z⁴ represent hydrogen and Z¹, W² and W³ represent methyl;
   (E) W¹, W², Z¹ and Z² represent hydrogen, W³ represents methyl and Z⁴ represents chloro;
(V) X¹ does not represent 4-chlorophenyl when:
   (A) W¹ and W³ represent methyl, W² represents hydrogen, and either: Z¹ and Z⁴ represent hydrogen and Z² represents chloro; Z¹ and Z² represent hydrogen and Z⁴ represents methyl; or Z² and Z⁴ represent hydrogen and Z¹ represents methyl;
   (B) W¹, W², Z¹ and Z⁴ represent hydrogen, and either: W³ represents ethyl and Z² represents chloro; or W³ represents methyl and Z² represents hydrogen;
   (C) W¹, W², Z¹ and Z² represent hydrogen, W³ represents isopropyl and Z⁴ represents methyl;
(VI) X¹ does not represent 3-nitrophenyl when W¹ and W³ represent methyl, W², Z¹ and Z⁴ represent hydrogen, and Z² represents chloro;

(g) W¹, W⁴, Z¹, Z² and Z³ all represent hydrogen, W² and W³ represent methyl, Z⁴ represents —OCH₃, then R does not represent 3-(methoxy)-4-(4-chlorobenzyloxy)-phenyl; and

(h) W¹, W³, W⁴, Z¹, Z², Z³ and Z⁴ represent hydrogen, W² represents X² in which X² represents —N(R^{3d})C(O)R^{4c}, R^{3d} represents hydrogen, then R and R^{3d} do not both represent 3-chlorophenyl, 4-methylphenyl, 4-chlorophenyl or unsubstituted phenyl,

which compounds are hereinafter referred to as ‘the compounds of the invention’.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallization. The various stereoisomers may be isolated by separation of a racemic or other mixture of the compounds using conventional, e.g. fractional crystallization or HPLC, techniques. Alternatively the desired optical isomers may be made by reaction of the appropriate optically active starting materials under conditions which will not cause racemization or epimerization (i.e. a ‘chiral pool’ method), by reaction of the appropriate starting material with a ‘chiral auxiliary’ which can subsequently be removed at a suitable stage, by derivatization (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an atom of the aromatic ring.

Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four and preferably, one to three) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzothiadiazolyl (including 2,1,3-benzothiadiazolyl), benzoxadiazolyl (including 2,1,3-benzoxadiazolyl), benzoxazinyl (including 3,4-dihydro-2H-1,4-benzoxazinyl), benzoxazolyl, benzimidazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are bicyclic or tricyclic, they are linked to the rest of the molecule via an atom of the aromatic ring. Heteroaryl groups may also be in the N- or S-oxidized form.

Heteroatoms that may be mentioned include phosphorus, silicon, boron, tellurium, selenium and, preferably, oxygen, nitrogen and sulfur.

For the avoidance of doubt, in cases in which the identity of two or more substituents in a compound of formula I may be the same, the actual identities of the respective substituents are not in any way interdependent. For example, in the situation in which W¹ and W² both represent X², then the respective X² groups in question may be the same or different. Similarly, when groups are substituted by more than one substituent as defined herein, the identities of those individual substituents are not to be regarded as being interdependent. For example, when R represents phenyl substituted by two X¹ groups in which one is R^{3a} and the other is —OR^{3h}, in which R^{3h} represents R^{3a}, and, in each case R^{3a} represents C₁₋₆ alkyl, the identities of the two R^{3a} groups are not to be regarded as being interdependent.

For the avoidance of doubt, when a term such as “W¹ to W⁴” is employed herein, this will be understood by the skilled person to mean W¹, W², W³ and W⁴ inclusively.

For the avoidance of doubt, where it is stated hereinbefore that at least one (e.g. one) X¹, X² or X³ group is present and represents a group containing at least one optionally substituted aryl or heteroaryl group, we mean that:

(i) R is substituted by at least one (e.g. one) of the relevant X¹ groups;

(ii) at least one (e.g. one) of W¹ to W⁴ represents a substituent selected from the relevant X² groups; and/or

(iii) at least one (e.g. one) of Z¹ to Z⁴ represents a substituent selected from the relevant X³ groups.

For the further avoidance of doubt, where it is stated that at least one X¹, X² or X³ group is present and represents a group containing an optionally substituted aryl or heteroaryl group, then:

R^{3b}, R^{3c}, R^{3h}, R³ⁱ, R^{3j}, R³ⁿ and/or R^{3p} (as appropriate) represent R^{3a}; and/or

at least one of R^{4a} and R^{5a}, R^{4b} and R^{5b}, R^{3d} and R^{4c}, R^{3e}, R^{4d}, and R^{5d}, R^{3f} and R^{4e}, R^{3g}, R^{4f} and R^{5f}, R^{4g} and R^{5g}, R^{3k} and R^{3m}, R^{4h} and R^{5h} and/o R⁴ⁱ and R⁵ⁱ (as appropriate) represent R^{3a}; and

R^{3a} represents aryl or heteroaryl (both of which are optionally substituted as defined above) or C₁₋₆ alkyl substituted by —N(R^{6b})R^{7b} or, more preferably, aryl, heteroaryl (both of which are optionally substituted as defined above) or —OR^{6a} in which R^{6a} and R^{6b} represent aryl or heteroaryl (optionally substituted as defined above; i.e. by G⁵) or C₁₋₆ alkyl substituted by aryl or heteroaryl (optionally substituted as defined above; i.e. by G⁶).

Hence, where it is stated that at least one X¹, X² or X³ group is present and represents a group containing an optionally substituted aryl or heteroaryl group (which may be referred to herein as the X¹, X² or X³ group containing the essential aryl or heteroaryl group), then we mean that that X¹, X² or X³ group contains an R^{3a} group that contains an aryl or heteroaryl group (optionally substituted as defined above). We refer to this group as the “R^{3a} group containing the essential aryl or heteroaryl group” (as defined in the paragraph above). Clearly, compounds of the invention can contain further X¹, X² or X³ groups that may also contain an R^{3a} group that contains an aryl or heteroaryl group, however, preferably, when further X¹, X² or X³ groups contain an R^{3a} group, then that R^{3a} group does not contain an optionally substituted aryl or heteroaryl group. We refer to this group as an “R^{3a} group that does not contain the essential aryl or heteroaryl group” (as defined in the paragraph below).

By an R^{3a} group that does not contain the essential aryl or heteroaryl group, we mean that:

R^{3a} represents C₁₋₆ alkyl optionally substituted by one or more substituents selected from F, Cl, ═O, —OR^{6a} and —N(R^{6b})R^{7b}; and

R^{6a} and R^{6b} independently represent H or C₁₋₆ alkyl optionally substituted by one or more substituents selected from F, Cl, ═O, —OR^{8a}, N(R^{9a})R^{10a} and —S(O)₂-G¹; and

R^{7b}, R^{8a}, R^{9a}, R^{10a} and G¹ are as hereinbefore defined.

Compounds of the invention that may be mentioned include those in which when Y represents —S(O)₂—, W¹, W², W³, W⁴, Z¹, Z² and Z³ represent hydrogen, R represents 4-methylphenyl, then Z⁴ does not represent 2-benzoxazolyl (this is referred to hereinafter as proviso (i)).

Compounds of the invention that may be mentioned include those in which at least one X¹, X² or X³ group is present and represents —R^{3a}, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p} or —S(O)₂N(R^{4h})R^{5h}, in which the foregoing groups contain at least one (e.g. one) aryl or heteroaryl group (both of which are optionally substituted as defined herein).

Preferred compounds of the invention that may be mentioned include those in which:

when R^{3a} represents C₁₋₆ alkyl substituted at the terminal position with two substituents, then those substituents cannot be both ═O and —OR^{6a} (so forming a —C(O)OR^{6a} group);

when, for example, any of W¹ to W⁴ (e.g. when one of W² or W³ represents X² and the other represents hydrogen) represent a substituent selected from X², then:

X² represents halo, —R^{3a}, —CN, —C(O)R^{3b}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ; and/or

R^{3a} represents, on each occasion when mentioned above, aryl, heteroaryl (which latter two groups are optionally substituted by one or more substituents selected from G³) or C₁₋₆ alkyl optionally substituted by one or more substituents selected from aryl, heteroaryl (which latter two groups are optionally substituted by one or more substituents selected from G⁴), F, Cl, ═O and —N(R^{6b})R^{7b};

R^{3a} represents, on each occasion when mentioned above, aryl, heteroaryl (which latter two groups are optionally substituted by one or more substituents selected from G³) or C₁₋₆ alkyl optionally substituted by one or more substituents selected from aryl, heteroaryl (which latter two groups are optionally substituted by one or more substituents selected from G⁴), F, Cl, —OR^{6a} and —N(R^{6b})R^{7b}; or

R^{3a} represents, on each occasion when mentioned above, aryl, heteroaryl (which latter two groups are optionally substituted by one or more substituents selected from G³) or C₁₋₆ alkyl optionally substituted by one or more substituents selected from aryl, heteroaryl (which latter two groups are optionally substituted by one or more substituents selected from G⁴), F, Cl and —N(R^{6b})R^{7b}; and/or

R^{6a} represents aryl, heteroaryl (which latter two groups are optionally substituted by one or more groups selected from G⁵) or C₁₋₆ alkyl optionally substituted by one or more substituents selected from aryl, heteroaryl (which latter two groups are optionally substituted by one or more groups selected from G⁶), F, Cl, ═O, —OR^{8a}, —N(R^{9a})R^{10a} and —S(O)₂-G¹; and/or

R^{8a} and R^{11a} independently represent —CH₃, —CH₂CH₃ or —CF₃; and/or

when R^{6a} represents optionally substituted C₁₋₆ alkyl, then those optional substituents are selected from either one of ═O and —OR^{8a}, and, more preferably, aryl, heteroaryl (which latter two groups are optionally substituted by one or more groups selected from G⁶), F, Cl, —N(R^{9a})R^{10a} and —S(O)₂-G¹.

Further preferred compounds of the invention that may be mentioned include those in which:

X¹, X² and X³ independently represent halo, —R^{3a}, —CN, —C(O)R^{3b}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ;

R^{3a} represents, on each occasion when mentioned above, aryl (which aryl group is optionally substituted by one or more substituents selected from G³) or C₁₋₆ alkyl optionally substituted by one or more substituents selected from either one of ═O and —OR^{6a} and, more preferably, aryl (which aryl group is optionally substituted by one or more substituents selected from G⁴), F, Cl and —N(R^{6b})R^{7b};

R^{6a} and R^{6b} independently represent H or, preferably, aryl (which aryl group is optionally substituted by one or more groups selected from G⁵) or C₁₋₆ alkyl optionally substituted by one or more substituents selected from either one of ═O and —OR^{8a} and, more preferably, aryl (which aryl group is optionally substituted by one or more groups selected from G⁶), F, Cl, —N(R^{9a})R^{10a} and —S(O)₂-G¹;

when R^{3a} represents heteroaryl or C₁₋₆ alkyl substituted by heteroaryl, or when R^{6a} or R^{6b} represent heteroaryl or C₁₋₆ substituted by heteroaryl, then preferably those heteroaryl groups contain one to three (e.g. one or two) heteroatoms.

Compounds of the invention that may be mentioned include those in which:

R represents optionally substituted phenyl, naphthyl, pyrrolyl, thienyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group;

when R represents a heteroaryl group, then it preferably represents:
(a) a heteroaryl group in which the heteroatom is sulfur or, preferably, nitrogen; and/or
(b) a 6 to 14-membered (e.g. 6- to 10-membered) heteroaryl group and, most preferably, a 6-membered heteroaryl group;

when R represents heteroaryl (e.g. furanyl such as 2-furanyl), substituted with X¹ (e.g. in the 5-position of the 2-furanyl group), then such a X¹ group preferably does not contain an R^{3a} group containing the essential aryl or heteroaryl group;

at least one of X² or, more preferably, X³ is present that contains the R^{3a} group containing the essential aryl or heteroaryl group;

when X² is present and contains the R^{3a} group containing the essential aryl or heteroaryl group, then preferably, W¹, W³ or W⁴ represent that X² group;

for example when W¹ to W⁴ (e.g. W²) represents X² that contains the R^{3a} group containing the essential aryl or heteroaryl group, then preferably, X¹, X² or X³ (e g. X²) represents —R^{3a}, —C(O)R^{3b}, —C(O)OR^{3c}, —C(O)N(R^{4a})R^{5a}, —N(R^{4b})R^{5b}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ.

Further preferred compounds of the invention that may be mentioned include those in which:

Y represents —S(O)₂—;

when Y represents —C(O)—, then:

at least one X³ group is present and represents —R^{3a}, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4c}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ, in which the foregoing groups contain at least one (e.g. one) aryl or heteroaryl group (both of which are optionally substituted as defined above), i.e. X³ is present that contains the R^{3a} group containing the essential aryl or heteroaryl group;

X¹ and X² do not represent —R^{3a}, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ, in which the foregoing groups contain an aryl or heteroaryl group, i.e. X¹ and X² (if present) do not contain an R^{3a} group containing the essential aryl or heteroaryl group;

X¹, preferably, X² or, more preferably, X³ represents the R^{3a} group containing the essential aryl or heteroaryl group;

the R^{3a} group containing the essential aryl or heteroaryl group represents C₁₋₆ alkyl substituted by one or more (e.g. one) substituent(s) selected from —N(R^{6b})R^{7b} and, preferably, aryl, heteroaryl (which latter two groups are optionally substituted by one or more substituents selected from G⁴) and —OR^{6a}, in which R^{6a} and R^{7b} represent aryl, heteroaryl (which latter two groups are optionally substituted by one or more substituents selected from G⁵) or C₁₋₆ alkyl optionally substituted by one or more substituents selected from aryl and heteroaryl (which latter two groups are optionally substituted by one or more substituents selected from G⁶);

when R represents phenyl, then that group is preferably substituted (e.g. in the ortho-position) by X¹, in which X¹ preferably represents a group that does not contain the essential aryl or heteroaryl group (i.e. it does not contain an R³ group that contains the essential aryl or heteroaryl group);

when X¹ represents a group that does not contain the essential aryl or heteroaryl group, then it preferably represents R^{3a} in which R^{3a} represents C₁₋₆ (e.g. C₁₋₃) alkyl (e.g. ethyl or, preferably, methyl) optionally substituted by one or more halo atoms (e.g. fluoro; so forming, for example, a difluoromethyl or, preferably, a trifluoromethyl group).

Preferred compounds of the invention include those in which:

when at least one of R^{3d} and R^{4c}, R^{3e}, R^{4d} and R^{5d}, R^{3f} and R^{4e}, R^{3g}, R^{4f} and R^{5f}, and R^{3k} and R^{3m} represent the R^{3a} group containing the essential aryl or heteroaryl group, then R^{4c}, R^{4e}, R^{3m}, R^{4d} or R^{5d} and R^{4f} or R^{5f} represents that R^{3a} group;

when at least one of R^{4a} and R^{5a}, R^{4b} and R^{5b}, R^{4d} and R^{5d}, R^{4f} and R^{5f}, R^{4g} and R^{5g}, R^{4h} and R^{5h}, and R⁴ⁱ and R⁵ⁱ represent the R^{3a} group containing the essential aryl or heteroaryl group, then only one of these represent that R^{3a} group.

Further compounds of the invention that may be mentioned include those in which:

when any one of W¹ to W⁴ (e.g. W² and/or W³) represents X², then X² does not represent —N(R^{4b})R^{5b} (e.g. when one of R^{4b} and R^{5b} is other than hydrogen).

Compounds of the invention that may be mentioned include those in which:

R does not represent pyrazolyl (e.g. 4-pyrazolyl; in which the ‘4’ represents the point of attachment of the pyrazolyl group to the rest of the compound of formula I);

when R represents pyrazolyl, it is preferably 5-pyrazolyl or, more preferably, 1- or 3-pyrazolyl (the skilled person will appreciate that it cannot represent 2-pyrazolyl due to the rules of valency).

Further compounds of the invention that may be mentioned include those in which:

at least one X² or X³ group is present and represents —R^{3a}, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ, in which the foregoing groups contain at least one (e.g. one) aryl or heteroaryl group (both of which are optionally substituted as defined above).

Further compounds of the invention that may be mentioned include those in which when R is substituted with X¹ and X¹ contains a R^{3a} group, then that R^{3a} group does not contain the essential aryl or heteroaryl group.

Compounds of the invention that may be mentioned include those in which:

when one of W² or W³ represents H, then the other does not represent tetrazolyl, —O-tetrazolyl, C₁₋₆ alkyl substituted by tetrazolyl or a C₁₋₆ alkyl group in which a —CH₂— group has been replaced with —O—, which (oxygen-containing C₁₋₆ ‘alkyl’) group is substituted by tetrazolyl.

Further compounds of the invention that may be mentioned include those in which when one of W² or W³ represents H, then the other does not represent:

R^{3a} in which R^{3a} represents tetrazolyl;

R^{3a} in which R^{3a} represents C₁₋₆ alkyl substituted by tetrazolyl;

R^{3a} in which R^{3a} represents C₁₋₅ alkyl substituted by —OR^{6a} in which R^{6a} represents tetrazolyl;

R^{3a} in which R^{3a} represents C₁₋₄ alkyl substituted by —OR^{6a} in which R^{6a} represents C₁₋₄ alkyl (in which the total number of carbon and oxygen atoms in the chain is not more than 6) substituted by tetrazolyl;

—OR^{3h} in which R^{3h} represents R^{3a} and R^{3a} is tetrazolyl;

—OR^{3h} in which R^{3h} represents R^{3a} and R^{3a} is C₁₋₅ alkyl substituted by tetrazolyl;

—OR^{3h} in which R^{3h} represents R^{3a} and R^{3a} is C₁₋₄ alkyl substituted by —OR^{6a} in which R^{6a} represents tetrazolyl;

—OR^{3h} in which R^{3h} represents R^{3a} and R^{3a} is C₁₋₄ alkyl substituted by —OR^{6a} in which R^{6a} represents C₁₋₄ alkyl (in which the total number of carbon and oxygen atoms in the chain is not more than 6) substituted by tetrazolyl.

Further compounds of the invention that may be mentioned include those in which:

when one of W² or W³ represents H, and the other represents X², then X² does not represent R^{3a} or —OR^{3h}, in which R^{3a} or R^{3h} contain a tetrazolyl group (e.g. when the total number of carbon and, where appropriate, oxygen atoms in R^{3a} or R^{3h} is no more than 6, not including the atoms of the tetrazole moiety).

Preferred compounds of the invention include those in which:

when any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, R⁴ⁱ and R⁵ⁱ and R^{6b} and R^{7b} are linked together, they form a 5- or 6-membered ring, which ring optionally contains a further heteroatom (such as nitrogen or oxygen) and is optionally substituted by R^{3a} (so forming, for example, a pyrrolidinyl, morpholinyl or a piperazinyl (e.g. 4-methylpiperazinyl) ring);

at least one (such as at least two (e.g. three)) of W¹ to W⁴ represent(s) hydrogen;

at least one (such as at least two) of Z¹ to Z⁴ represent(s) hydrogen;

R is substituted with less than four (e.g. less than three) X¹ substituents;

R^{3c} represents R^{3a};

R^{3j} (e.g. when m represents 1 or 2) represents R^{3a};

X¹, X² or X³ independently represent —OS(O)₂N(R⁴ⁱ)R⁵ⁱ or, preferably, halo (e.g. fluoro, bromo or, particularly, chloro), R^{3a}, —CN, —C(O)N(R^{4a})R^{5a}, —N(R^{4b})R^{5b}, —N(R^{3d})C(O)R^{4c}, —OR^{3h}, —S(O)ₘR^{3j} or —S(O)₂N(R^{4h})R^{5h};

m represents 2;

when R^{3j} represents R^{3a}, then R^{3a} preferably represents C₁₋₃ alkyl (e.g. methyl or ethyl);

R^{4b}, R^{5b}, R^{4h}, R^{5h}, R⁴ⁱ and R⁵ⁱ independently represent H or C₁₋₂ alkyl (e.g. methyl); or

R^{4b} and R^{5b}, R^{4h} and R^{5h} or R⁴ⁱ and R⁵ⁱ are linked together as herein described;

R³ⁿ represents R^{3a};

when R³ⁿ represents R^{3a}, then R^{3a} preferably represents C₁₋₃ alkyl (e.g. methyl or trifluoromethyl);

R^{6a} and R^{6b} independently represent H, aryl (such as phenyl, optionally substituted by one or more G⁵ groups) or C₁₋₆ alkyl optionally substituted by one or more fluoro atoms;

R^{7b} represents H or C₁₋₆ alkyl optionally substituted by one or more fluoro atoms;

G¹ and G² independently represent —N(R^{14a})R^{15a} or C₁₋₃ alkyl (e.g. methyl) optionally substituted by one or more chloro or, preferably, fluoro atoms (so forming, for example, a trifluoromethyl group);

G³, G⁴, G⁵ and G⁶ independently represent halo, R^{20a} or —OR^{20h};

R^{20c} represents R^{20a};

R^{20j} (e.g. when m represents 1 or 2) represents R^{20j};

R^{20a} represent C₁₋₆ (e.g. C₁₋₃) alkyl optionally substituted by one or more T¹ substituents;

T¹ and T² independently represent Cl or, preferably, F;

R^{23a}, R^{23b} and R^{24b} independently represent H or C₁₋₃ alkyl optionally substituted by one or more T³ substituents;

T³ and T⁴ independently represent F;

R^{25a}, R^{25b} and R^{26b} independently represent H, —CH₃ or —CF₃.

Preferred aryl and heteroaryl groups that R may represent (or that the aryl or heteroaryl groups of the R^{3a} groups containing the essential aryl or heteroaryl group may represent) include optionally substituted (e.g. in the case of R, by X¹) phenyl, naphthyl, pyrrolyl, furanyl, thienyl (e.g. thien-2-yl or thien-3-yl), imidazolyl (e.g. 1-imidazolyl, 2-imidazolyl or 4-imidazolyl), oxazolyl, isoxazolyl, thiazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl (e.g. 2-pyrazinyl), indazolyl, benzimidazolyl, quinazolinyl, quinoxalinyl, 1,3-benzodioxolyl, tetrazolyl, benzothiazolyl, and/or benzodioxanyl, group. Preferred values include phenyl.

Preferred compounds of the invention include those in which:

R represents phenyl optionally substituted by one or two X¹ substituents;

W¹ and W⁴ independently represent H;

W² and W³ independently represent X² or H;

one of W¹ to W⁴ (e.g. W² or W³) represents X² and the others represent H;

Z¹ represents H;

Z², Z³ and Z⁴ independently represent X³ or H;

all of Z¹ to Z⁴ represent H; or

one of Z¹ to Z⁴ (e.g. Z², Z³ or Z⁴) or two of Z¹ to Z⁴ (e.g. Z³ and Z⁴) represent(s) X³ and the others represent H;

only one X¹, X² or X³ (e.g. X² or, more preferably, X³) group is present in which it contains an R^{3a} group containing the essential aryl or heteroaryl group, and when other X¹, X² or X³ groups are present, then they preferably do not represent a group that contains an R³a group containing the essential aryl or heteroaryl group.

Preferred compounds of the invention that may be mentioned include those in which:

X¹, X² or X³ independently represent halo (e.g. chloro), R^{3a}, —C(O)N(R^{4a})R^{5a}, —N(R^{4b})R^{5b}—N(R^{3d})C(O)R^{4c} or —OR^{3h};

R^{4a} represents H or an R^{3a} group that does not contain the essential aryl or heteroaryl group;

R^{5a} represents R^{3a};

R^{4b} and R^{5b} independently represent hydrogen;

R^{3d} represents H;

R^{4c} represents R^{3a};

R^{3h} represents R^{3a};

R^{3a} represents aryl, such as phenyl (optionally substituted by one substituent selected from G³), C₁₋₆ (e.g. C₁₋₄) alkyl (e.g. methyl, propyl, propenyl (such as —CH₂—CH═CH—) or tert-butyl) optionally substituted by one or more fluoro atoms (so forming, for example, a trifluoromethyl group), aryl (e.g. phenyl) groups (optionally substituted as defined herein, i.e. by G⁴, or, preferably, unsubstituted) or —OR^{6a} groups;

R^{6a} represents phenyl optionally substituted by G⁵;

G³ represents halo (e.g. chloro), R^{20a} or —OR^{20h};

R^{20h} represents R^{10a},

R^{20a} represents C₁₋₃ alkyl (e.g. methyl or isopropyl);

G⁴, G⁵ and G⁶ (e.g. G⁵) independently represent halo (e.g. fluoro or, preferably, chloro).

Further preferred compounds of the invention include those in which:

when any one of X¹, X² or X³ contain an R^{3a} group containing the essential aryl or heteroaryl group, then they independently represent R^{3a}, —C(O)N(R^{4a})R^{5a}, —N(R^{3d})C(O)R^{4c} or —OR^{3h}; and

R^{3a} represents aryl, such as phenyl, (optionally substituted by one substituent selected from G³), C₁₋₃ alkyl (e.g. methyl, propyl or propenyl (such as —CH₂—CH═CH—)) substituted by phenyl or —OR^{6a}; or

when any one of X¹, X² or X³ contain an R^{3a} group that does not contain the essential aryl or heteroaryl group, then they preferably represent halo (e.g. chloro), R^{3a}, —N(R^{4b})R^{5b} or —OR^{3h}; and

R^{3a} represents C₁₋₄ alkyl (e.g. methyl or tert-butyl) optionally substituted by one or more fluoro atoms (so forming, for example, a trifluoromethyl group).

Most preferred compounds of the invention include those in which:

when X¹ contains an R^{3a} group that does not contain the essential aryl or heteroaryl group, then it preferably represents —OCF₃, —CF₃ or chloro;

when X² contains an R^{3a} group containing the essential aryl or heteroaryl group, then it preferably represents —N(H)C(O)-[3,5-dichlorophenyl] or 4-isopropyl-phenyl;

when X² contains an R^{3a} group that does not contain the essential aryl or heteroaryl group, then it preferably represents methyl or tert-butyl;

when X³ contains an R^{3a} group containing the essential aryl or heteroaryl group, then it preferably represents 3,5-dimethylphenyl, —O—CH₂-phenyl (i.e. benzyloxy), —C(O)—N(CH₃)-[3-chloro-2-methylphenyl], —C(O)—N(H)-[3-chloro-2-methylphenyl], —O-(4-chlorophenyl) (i.e. 4-chlorophenoxy), —O—CH₂—CH═CH-phenyl, —O—(CH₂)₃-phenyl, —CH₂—O-(4-chlorophenyl), —CH₂—O-(3-chlorophenyl) or —CH₂—O-(2-chlorophenyl);

when X³ contains an R^{3a} group that does not contain the essential aryl or heteroaryl group, then it preferably represents chloro or amino (e.g. —NH₂).

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I, which process comprises:

(i) reaction of a compound of formula II,

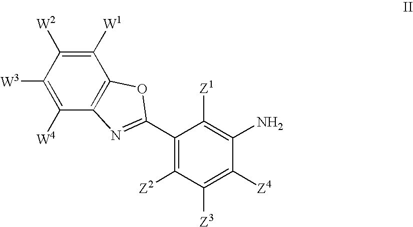

wherein W¹ to W⁴ and Z¹ to Z⁴ are as hereinbefore defined, with a compound of formula III,

R—Y—OH III

wherein R and Y are as hereinbefore defined, under coupling conditions, for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, trifluoromethylbenzene, triethylamine or water) and a suitable coupling agent (e.g. 1,1′-carbonyldiimidazole, N,N′-dicyclohexylcarbodiimide, 1-(3-dimethylamino-propyl)-3-ethylcarbodiimide (or hydrochloride thereof), N,N′-disuccinimidyl carbonate, benzotriazol-1-yloxytris(dimethylamino)phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluorophosphate, bromo-tris-pyrrolidinophosponium hexafluorophosphate, 2-(1H-benzotriazol-1-yl)-1,1,3,3-tetramethyluronium tetrafluorocarbonate) or 1-cyclohexylcarbodiimide-3-propyloxymethyl polystyrene). Alternatively, compounds of formula III may first be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride. This activated intermediate may then be reacted with a compound of formula II under standard conditions, such as those described above. Alternatively, an azodicarboxylate may be employed under Mitsunobo conditions known to those skilled in the art; or

(ii) reaction of a compound of formula IV,

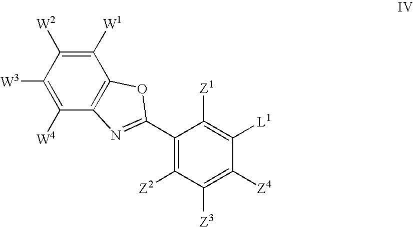

wherein L¹ represents a suitable leaving group, such as chloro, bromo, iodo, a sulfonate group (e.g. —OS(O)₂CF₃, —OS(O)₂CH₃, —OS(O)₂PhMe or a nonaflate) or —B(OH)₂ and W¹ to W⁴ and Z¹ to Z⁴ are as hereinbefore defined, with a compound of formula V,

H₂N—Y—R V

wherein R and Y are as hereinbefore defined, for example optionally in the presence of an appropriate metal catalyst (or a salt or complex thereof) such as Cu, Cu(OAc)₂, CuI (or CuI/diamine complex), Pd(OAc)₂, Pd₂(dba)₃ or NiCl₂ and an optional additive such as Ph₃P, 2,2′-bis(diphenylphosphino)-1,1′-binaphthyl, xantphos, NaI, or an appropriate crown ether such as 18-crown-6-benzene, in the presence of an appropriate base such as NaH, Et₃N, pyridine, N,N′-dimethylethylenediamine, Na₂CO₃, K₂CO₃, DABCO, K₃PO₄, Cs₂CO₃, t-BuONa or t-BuOK (or a mixture thereof), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent. This reaction may be carried out at room temperature or above (e.g. at a high temperature, such as the reflux temperature of the solvent system that is employed) or using microwave irradiation.

Compounds of formula II may be prepared by reduction of a compound of formula VI,

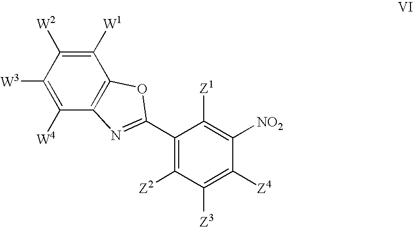

wherein W¹ to W⁴ and Z¹ to Z⁴ are as hereinbefore defined, under standard conditions known to those skilled in the art. For example, the reduction may be performed by hydrogenation (e.g. catalytic hydrogenation (e.g. employing 10% Pd/C)), for example in the presence of an alcoholic solvent (e.g. EtOH), or in the presence of other suitable reducing conditions, such as employing a mixture of Sn/HCl or Fe powder in EtOH and NH₄Cl.

Compounds of formula II in which either one of X² or X³ (e.g. X³) is present and represents R^{3a}, and R^{3a} represents C₁₋₆ alkyl substituted by F, Cl, —OR^{6a} (in which R^{6a} does not represent H) or —N(R^{6b})R^{7b}, may be synthesized by reaction of a corresponding compound of formula II in which R^{3a} represents C₁₋₆ alkyl substituted by —OR^{6a} in which R^{6a} represents H (or a compound corresponding to such a compound but in which the relevant —OH group is replaced with a suitable leaving group, for example chloro, bromo, iodo or a sulfonate group such as —OS(O)₂CF₃, —OS(O)₂CH₃, —OS(O)₂PhMe or a nonaflate) with a compound of formula VIA,

HR^{z} VIA

wherein R^{z} represents F, Cl, —OR^{6ab} or —N(R^{6b})R^{7b}, in which R^{6ab} represents R^{6a} provided that it does not represent H, and R^{6a}, R^{6b} and R^{7b} are as hereinbefore defined, under standard reaction conditions. For example, for reaction with a compound of formula II in which the R^{3a} group is C₁₋₆ alkyl substituted by —OH, under Mitsunobu reaction conditions known to those skilled in the art, for example in the presence of Ph₃P (or the like) and an azo dicarboxylate (e.g. DIAD or DEAD, or the like), for example in the presence of a suitable solvent at or below room temperature (e.g. at about 0° C). For reaction with a compound corresponding to a compound of formula II in which the R^{3a} group is C₁₋₆ alkyl substituted by —OH, but in which the —OH group is replaced with a suitable leaving group, under standard conditions, for example in the presence of a suitable base and solvent system (such as those described hereinbefore in respect of preparation of compounds of formula I (process step (i)).

Compounds of formula II in which any one of X¹, X² or X³ (e.g. X³) represents R^{3a}, and R^{3a} represents C₁₋₆ alkyl substituted by a —OH group may be synthesized from a corresponding compound of formula II in which R^{3a} represents C₁₋₆ alkyl substituted by a —OR^{6a} group and a ═O substituent that is α to that —OR^{6a} group, under standard reduction conditions known to those skilled in the art. For example, in the case where R^{6a} represents H (so forming a —C(O)OH group), reduction may be performed in the presence of borane (or a source thereof, such as a molar THF-complex solution), for example in a suitable solvent (e.g. THF), or in the case where R^{6a} is other than H (so forming an ester), reduction may be performed in the presence of borane or, preferably, in the presence of LiAlH₄ or LiBH₄, for example under similar conditions to those described above in respect of the reduction of the carboxylic acid group.

Compounds of formula IV (e.g. those in which L¹ represents halo, and preferably bromo) in which Z¹ to Z⁴ (e.g. Z² or Z³) represent R^{3a}, in which R^{3a} is an aryl or heteroaryl (optionally substituted as described herein), may be prepared from a corresponding compound of formula IV in which that Z¹ to Z⁴ group represents H, with a compound of formula VIB,

L^{x}R^{3ab} VIB

wherein L^{x} represents a suitable leaving group such as halo (e.g. iodo) and R^{3ab} represents aryl or heteroaryl optionally substituted by one or more substituents selected from G³, under standard coupling conditions, for example such as those hereinbefore described in respect of preparation of compounds of formula I (process step (ii)) or employing a metal catalyst as defined therein (e.g. Pd(OAc)₂) together with AgOAc, for example in a solvent such as trifluoroacetic acid and which reaction may be preformed at elevated temperature (e.g. at about 180° C.), for example in a sealed pressure resistant vessel.

Compounds of formula IV in which X² or X³ represent —OR^{3h} in which R^{3h} is other than H may be prepared by reaction of corresponding compounds of formula IV in which R^{3h} is H with a compound of formula VIC,

R^{3ha}L^{z} VIC

wherein L^{z} represents a suitable leaving group such as halo (e.g. bromo or chloro) and R^{3ha} represents R^{3h} provided that it does not represent H, under standard alkylation conditions, for example at around room temperature, below room temperature (e.g. at 0° C.) or above room temperature (e.g. up to 60-70° C.) optionally in the presence of a suitable base (e.g. sodium hydride, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, K₂CO₃, or mixtures thereof) and an appropriate solvent (e.g. dimethylformamide, pyridine, dichloromethane, chloroform, tetrahydrofuran, dimethylsulfoxide, acetonitrile, water, or mixtures thereof), optionally under inert (e.g. anhydrous) conditions.

Compounds of formulae II, IV and VI may be prepared by:

(I) reaction of a compound of formula VII,

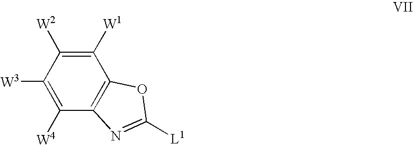

wherein L¹ and W¹ to W⁴ are as hereinbefore defined, with a compound of formula VIII,

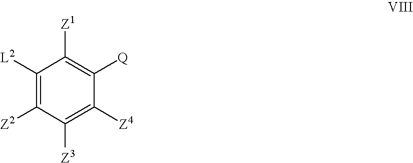

wherein L² represents a suitable leaving group such as chloro, bromo, iodo, —B(OH)₂ or a protected derivative thereof, for example a 4,4,5,5-tetramethyl-1,3,2-dioxaborolan-2-yl group, 9-borabicyclo[3.3.1]nonane (9-BBN), —Sn(alkyl)₃ (e.g. —SnMe₃ or —SnBu₃), or a similar group known to the skilled person, Q represents —NH₂ (for preparation of compounds of formula II), L¹ (for preparation of compounds of formula IV) or —NO₂ (for preparation of compounds of formula VI), as appropriate, and Z¹ to Z⁴ are as hereinbefore defined. The skilled person will appreciate that L¹ and L² will be mutually compatible, and that both must be compatible with Q (e.g. when Q is —NH₂) in compounds of formula VIII. This reaction may be performed, for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-binaphthyl, 1,1′-bis(diphenyl-phosphinoferrocene), 1,3-bis(diphenylphosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation;

(II) reaction of a compound of formula IX,

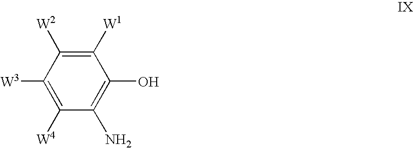

wherein W¹ to W⁴ are as hereinbefore defined, with a compound of formula X,

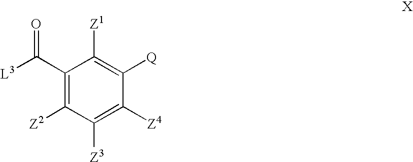

wherein L³ represents a suitable leaving group, such as chloro, bromo, or a hydroxy group, which latter group may be activated by employing a suitable reagent such as one defined hereinbefore in respect of preparation of compounds of formula I (process step (i) above), and Q and Z¹ to Z⁴ are as hereinbefore defined, for example under reaction conditions such as those described hereinbefore in respect of preparation of compounds of formula I (process step (i) above), followed by standard condensation/dehydration conditions. The skilled person will appreciate that this reaction step may proceed via intermediates such as compounds of formula XI or XII described hereinafter;

(III) intramolecular reaction of a compound of formula XI,

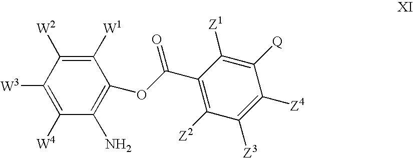

wherein W¹ to W⁴, Z¹ to Z⁴ and Q are as hereinbefore defined or a compound of formula XII,

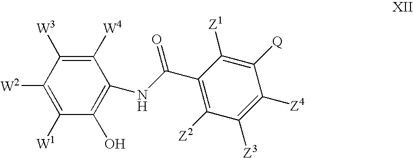

wherein W¹ to W⁴, Z¹ to Z⁴ and Q are as hereinbefore defined, both of which may be allowed to react under reaction conditions known to those skilled in the art, for example standard cyclization conditions, followed by standard condensation/dehydration conditions; or

(IV) either:
(a) preparing, from a compound of formula VII in which L¹ represents halo:
   (1) a corresponding magnesium-containing reagent (e.g. Grignard reagent) under standard conditions known to those skilled in the art; or
   (2) a corresponding lithiated compound under halogen-lithium exchange reaction conditions known to those skilled in the art; or
(b) preparing, from a compound corresponding to a compound of formula VII but in which L¹ represents H, a compound corresponding to a compound of formula VII but in which L¹ is lithium, under appropriate lithiation conditions,

and then reacting the resultant intermediate with a compound of formula VIII in which L² represents a suitable leaving group such as bromo, for example under conditions such as those described hereinbefore in respect of preparation of compounds of formulae II, IV or VI (process step (I) above). The skilled person will also appreciate that the magnesium of the magnesium-containing reagent (e.g. Grignard reagent) or the lithium of the lithiated species may be exchanged (and, in the case of the lithiated species, is preferably exchanged) to a different metal (i.e. a transmetallation reaction may be performed), for example to zinc (e.g. using ZnCl₂) and the intermediate so formed may then be subjected to reaction with a compound of formula VIII, for example under reaction conditions described above.

Compounds of formula IX in which W¹ to W⁴ represent X² in which X² represents R^{3a} and R^{3a} is an aryl or heteroaryl group (optionally substituted as described herein) may be prepared by reaction of a corresponding compound of formula IX in which that X² group represents halo (e.g. iodo or preferably, bromo) with a compound of formula XIII,

L^{y}R^{3ab} XIII

wherein L^{y} represents a suitable leaving group, such as chloro, bromo, iodo, a sulfonate group (e.g. —OS(O)₂CF₃, —OS(O)₂CH₃, —OS(O)₂PhMe or a nonaflate) or —B(OH)₂ and R^{3ab} is as defined above, for example under similar conditions to those described hereinbefore in respect of preparation of compounds of formula I (process (ii)).

Compounds of formulae III, V, VIA, VIB, VIC, VII, VIII, IX, X, XI, XII and XIII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents W¹ to W⁴, Z¹ to Z⁴ and optional substituents on R in final compounds of formula I or relevant intermediates may be modified one or more times, after or during the processes described above by way of methods that are well known to those skilled in the art. Examples of such methods include substitutions, reductions (e.g. of double bonds to single bonds by hydrogenation), oxidations, alkylations, acylations, hydrolyses, esterifications, and etherifications. The precursor groups can be changed to a different such group, or to the groups defined in formula I, at any time during the reaction sequence. In this respect, the skilled person may also refer to “Comprehensive Organic Functional Group Transformations” by A. R. Katritzky, O. Meth-Cohn and C. W. Rees, Pergamon Press, 1995.

For example, in the case where R¹ or R² represents a halo group, such groups may be inter-converted one or more times, after or during the processes described above for the preparation of compounds of formula I. Appropriate reagents include NiCl₂ (for the conversion to a chloro group). Further, oxidations that may be mentioned include oxidations of sulfanyl groups to sulfoxide and sulfonyl groups, for example employing standard reagents (e.g. meta-chloroperbenzoic acid, K₂MnO₄ or a solution of Oxone® in ethylenediaminetetraacetic acid).

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of formula I may be isolated from their reaction mixtures using conventional techniques.

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3rd edition, T. W. Greene & P. G. M. Wutz, Wiley-Interscience (1999).

Medical and Pharmaceutical Uses

Compounds of the invention are indicated as pharmaceuticals. According to a further aspect of the invention there is provided a compound of the invention, as hereinbefore defined but without provisos (b), (d) to (h) (inclusive; i.e. provisos (d), (e), (f), (g) and (h)) and, if applicable, (i) for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolized in the body to form compounds of the invention. Such compounds (which may possess some pharmacological activity, provided that such activity is appreciably lower than that of the “active” compounds to which they are metabolized) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention may possess no or minimal pharmacological activity as such, but may be administered parenterally or orally, and thereafter be metabolized in the body to form compounds (e.g. compounds of the invention) that possess pharmacological activity as such. Such compounds (which also includes compounds that may possess some pharmacological activity, but that activity is appreciably lower than that of the “active” compounds of the invention to which they are metabolized), may also be described as “prodrugs”.

Thus, the compounds of the invention are useful because they possess pharmacological activity, and/or are metabolized in the body following oral or parenteral administration to form compounds which possess pharmacological activity (e.g. similar or pronounced pharmacological activity as compared to the compounds of the invention from which they are formed).

Compounds of the invention are particularly useful because they may inhibit the activity of a member of the MAPEG family.

Compounds of the invention are particularly useful because they may inhibit (for example selectively) the activity of prostaglandin E synthases (and particularly microsomal prostaglandin E synthase-1 (mPGES-1)), i.e. they prevent the action of mPGES-1 or a complex of which the mPGES-1 enzyme forms a part, and/or may elicit a mPGES-1 modulating effect, for example as may be demonstrated in the test described below. Compounds of the invention may thus be useful in the treatment of those conditions in which inhibition of a PGES, and particularly mPGES-1, is required.

Compounds of the invention are thus expected to be useful in the treatment of inflammation.

The term “inflammation” will be understood by those skilled in the art to include any condition characterized by a localized or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterized by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Accordingly, compounds of the invention may be useful in the treatment of asthma, chronic obstructive pulmonary disease, pulmonary fibrosis, inflammatory bowel disease, irritable bowel syndrome, inflammatory pain, fever, migraine, headache, low back pain, fibromyalgia, myofascial disorders, viral infections (e.g. influenza, common cold, herpes zoster, hepatitis C and AIDS), bacterial infections, fungal infections, dysmenorrhea, burns, surgical or dental procedures, malignancies (e.g. breast cancer, colon cancer, and prostate cancer), hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, neurodegenerative disorders such as Alzheimer's disease and multiple sclerosis, autoimmune diseases, allergic disorders, rhinitis, ulcers, coronary heart disease, sarcoidosis and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds the invention may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a member of the MAPEG family such as a PGES (e.g. mPGES-1), LTC₄ and/or FLAP and/or a method of treatment of a disease in which inhibition of the activity of a member of the MAPEG family such as PGES (and particularly mPGES-1), LTC₄ and/or FLAP is desired and/or required (e.g. inflammation), which method comprises administration of a therapeutically effective amount of a compound of the invention, as hereinbefore defined but without the provisos (e.g. without provisos (b) to (i)), to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without provisos (b), (d) to (h) and, if applicable, (i), in admixture with a pharmaceutically acceptable adjuvant, diluent or carrier.

Preferred pharmaceutical formulations include those in which the active ingredient is present in at least 1% (such as at least 10%, preferably in at least 30% and most preferably in at least 50%) by weight. That is, the ratio of active ingredient to the other components (i.e. the addition of adjuvant, diluent and carrier) of the pharmaceutical composition is at least 1:99 (e.g. at least 10:90, preferably at least 30:70 and most preferably at least 50:50) by weight.

The invention further provides a process for the preparation of a pharmaceutical formulation, as hereinbefore defined, which process comprises bringing into association a compound of the invention, as hereinbefore defined but without provisos (b), (d) to (h) and, if applicable, (i), or a pharmaceutically acceptable salt thereof with a pharmaceutically-acceptable adjuvant, diluent or carrier.

Compounds of the invention may also be combined with other therapeutic agents that are useful in the treatment of inflammation (e.g. NSAIDs and coxibs).

According to a further aspect of the invention, there is provided a combination product comprising:
(A) a compound of the invention, as hereinbefore defined but without the provisos (e.g. without proviso (c) and, particularly, without provisos (b) and (d) to (i)); and
(B) another therapeutic agent that is useful in the treatment of inflammation, wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without the provisos (e.g. without proviso (c) and, particularly, without provisos (b) and (d) to (i)), another therapeutic agent that is useful in the treatment of inflammation, and a pharmaceutically-acceptable adjuvant, diluent or carrier; and
(2) a kit of parts comprising components:
(a) a pharmaceutical formulation including a compound of the invention, as hereinbefore defined but without the provisos (e.g. without proviso (c) and, particularly, without provisos (b) and (d) to (i)), in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,

which components (a) and (b) are each provided in a form that is suitable for administration in conjunction with the other.

The invention further provides a process for the preparation of a combination product as hereinbefore defined, which process comprises bringing into association a compound of the invention as hereinbefore defined but without the provisos (e.g. without proviso (c) and, particularly, without provisos (b) and (d) to (i)) with another therapeutic agent that is useful in the treatment of inflammation, and a pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective, and preferably selective, inhibitors of a member of MAPEG family, e.g. inhibitors of prostaglandin E synthases (PGES) and particularly microsomal prostaglandin E synthase-1 (mPGES-1). The compounds of the invention may reduce the formation of the specific arachidonic acid metabolite PGE₂ without reducing the formation of other COX generated arachidonic acid metabolites, and thus may not give rise to the associated side-effects mentioned hereinbefore.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

Biological Test

In the assay mPGES-1 catalyzes the reaction where the substrate PGH₂ is converted to PGE₂. mPGES-1 is expressed in E. coli and the membrane fraction is dissolved in 20 mM NaPi-buffer pH 8.0 and stored at −80° C. In the assay mPGES-1 is dissolved in 0.1M KPi-buffer pH 7.35 with 2.5 mM glutathione. The stop solution consists of H₂O/MeCN (7/3), containing FeCl₂ (25 mM) and HCl (0.15 M). The assay is performed at room temperature in 96-well plates. Analysis of the amount of PGE₂ is performed with reversed phase HPLC (Waters 2795 equipped with a 3.9×150 mm C18 column). The mobile phase consists of H₂O/MeCN (7/3), containing TFA (0.056%), and absorbance is measured at 195 nm with a Waters 2487 UV-detector.

The following is added chronologically to each well:
1. 100 μL mPGES-1 in KPi-buffer with glutathione. Total protein concentration: 0.02 mg/mL.
2. 1 μL inhibitor in DMSO. Incubation of the plate at room temperature for 25 minutes.
3. 4 μL of a 0.25 mM PGH₂ solution. Incubation of the plate at room temperature for 60 seconds.
4. 100 μL stop solution.
   180 μL per sample is analyzed with HPLC.

### EXAMPLES

The invention is illustrated by way of the following examples, in which the following abbreviations may be employed:

aq. aqueous

DMAP 4-dimethylaminopyridine

DMF dimethylformamide

DMSO dimethylsulfoxide

EtOAc ethyl acetate

NMR nuclear magnetic resonance

THF tetrahydrofuran

### Example 1

### 3,5-Dichloro-N-(2-{3-[(3,5-dichlorobenzoyl)amino]phenyl}-1,3-benzoxazol-6-yl)benzamide

### (a) 6-Nitro-2-(3-nitrophenyl)benzoxazole

A mixture of 2-amino-5-nitrophenol (e.g. 18 mmol) and 3-nitrobenzoyl chloride (e.g. 20 mmol) in 1,4-dioxane (e.g. 25 mL) was placed in microwave process vials and each of the sealed reaction vessel was treated with microwaves (e.g. for 15 min at 210° C.). After cooling, the reaction mixture was poured into to a stirred solution of 1N NaOH (e.g. 300 mL), yellow precipitate filtered and dried in vacuo to afford the sub-title compound.

### (b) 2-(3-Aminophenyl)benzoxazol-6-ylamine

A solution of 6-nitro-2-(3-nitrophenyl)benzoxazole (e.g. 11.9 mmol; see step (a) above) in glacial acetic acid (75 mL) was hydrogenated (e.g. under 4 atm of hydrogen pressure in presence of 10% Pd—C (e.g. 127 mg, 1.19 mmol) at room temperature for 4 hours). After filtration through a celite, solvent was evaporated, the residue dissolved in EtOAc (e.g. 100 mL) and washed with aq. saturated NaHCO₃. Drying and filtration through a silica gel pad afforded the sub-title compound.

### (c) 3,5-Dichloro-N-(2-{3-[(3,5-dichlorobenzoyl)amino]phenyl}-1,3-benzoxazol-6-yl)benzamide

A mixture of 2-(3-aminophenyl)benzoxazol-6-ylamine (e.g. 2.5 mmol; see step (b) above) and 3,5-dichlorobenzoyl chloride (685 mg, 3.75 mmol) in toluene (e.g. 25 mL) was heated (e.g. under reflux for 1.5 hours), cooled and filtered. Solid was recrystallized (e.g. from ethyl alcohol) to afford the title compound. The filtrate may be concentrated and the solid residue may be recrystallized (e.g. from ethyl alcohol) to yield additional solid title compound.

200 MHz ¹H-NMR (DMSO-d₆, ppm) δ 10.68 (1H, s) 10.64 (1H, s) 8.67 (1H, dd, J=1.6, 1.6 Hz) 8.33-8.32 (1H, m) 8.04-7.86 (8H, m) 7.78 (1H, d, J=8.8 Hz) 7.66 (1H, dd, J=8.8, 1.4 Hz) 7.58 (1H, dd, J=8.0, 8.0 Hz).

### Example 2

### N-[3′,5′-Dimethyl-2-(5-methylbenzoxazol-2-yl)biphenyl-4-yl3-2-trifluoromethoxy-benzamide

### (a) 2-(3-Bromophenyl)-5-methyl-benzoxazole

The sub-title compound was prepared in accordance with Example 1, step (a) from 2-amino-4-methylphenol and 3-bromobenzoyl chloride.

### (b) 2-(4-Bromo-3′,5′-dimethylbiphenyl-2-yl)-5-methylbenzoxazole

An ACE® pressure tube was charged with 2-(3-bromophenyl)-5-methylbenzoxazole (548 mg, 1.9 mmol; see step (a) above), 1-iodo-3,5-dimethylbenzene (1.1 mL, 7.6 mmol), Pd(OAc)₂ (12.8 mg, 0.057 mmol), AgOAc (317 mg, 1.9 mmol) and trifluoroacetic acid (4 mL) and the resulting mixture was heated at 180° C. for 84 h. After cooling, the reaction mixture was poured into water and extracted with MeOtBu (3×50 mL). The combined organic extracts were washed with aqueous saturated NaHCO₃, brine and then dried over Na₂SO₄. Concentration under reduced pressure and purification by chromatography afforded the sub-title compound (390 mg, 52%).

### (c) N-[3′,5′-Dimethyl-2-(5-methylbenzoxazol-2-yl)biphenyl-4-yl]-2-trifluoromethoxybenzamide

2-(4-Bromo-3′,5′-dimethylbiphenyl-2-yl)-5-methylbenzoxazole (e.g. 0.50 mmol; see step (b) above), CuI (e.g. 0.06 mmol), K₃PO₄ (e.g. 1.2 mmol), N,N′-dimethyl-1,2-diaminoethane (e.g. 0.18 mmol) and 2-trifluoromethoxybenzamide (e.g. 0.5 mmol) in toluene (e.g. 2 mL) was heated (e.g. at 110° C. for 48 h). The mixture was diluted with EtOAc (70 mL), filtered through a celite and dried over Na₂SO₄. Solvent removal and recrystallization from DMF afforded the title compound.

200 MHz ¹H-NMR (DMSO-d₆, ppm) δ 10.82 (1H, s) 8.44 (1H, d, J=2.2 Hz) 7.91 (1H, dd, J=8.4, 2.2 Hz) 7.79-7.47 (6H, m) 7.37 (1H, d, J=8.4 Hz) 7.14 (1H, dd, J=8.4, 1.4 Hz) 6.93 (1H, br s) 6.81 (2H, br s) 2.39 (3H, s) 2.16 (6H, s).

### Example 3

### N-[4-Benzyloxy-3-(5-methylbenzoxazol-2-yl)phenyl]-2-trifluoromethylbenzamide

### (a) 4-Bromo-2-(5-methylbenzoxazol-2-yl)phenol

A mixture of 2-amino-4-methylphenol (18 mmol, 2.22 g) and 5-bromo-2-hydroxy-benzoyl chloride (20 mmol, 4.69 g) in 25 mL of 1,4-dioxane was placed in 10 microwave process vials and each of the sealed reaction vessels was treated with microwaves for 15 min at 210° C. After cooling, the reaction mixture was filtered through Celite®. The filter cake was washed with EtOAc. The combined filtrates were concentrated and purified by chromatography to give the sub-title compound (3.91 g, 72%).

### (b) 2-(2-Benzyloxy-5-bromophenyl)-5-methylbenzoxazole

A solution of 4-bromo-2-(5-methylbenzoxazol-2-yl)phenol (e.g. 3.29 mmol; see step (a) above) in dry DMF (e.g. 30 mL) was added gradually to a suspension of 75% NaH (e.g. 3.62 mmol; washed twice with dry Et₂O prior to use) in DMF (e.g. 5.0 mL) at 0° C. The reaction mixture was stirred at 0° C. for 30 min, whereupon chloromethylbenzene (e.g. 6.58 mmol) in DMF (15 mL) was added. After stirring at room temperature for 24 h, the mixture was poured in water (e.g. 100 mL) and extracted with MeOtBu. The combined extracts were washed with water and brine and then dried over Na₂SO₄. Concentration under reduced pressure and purification by chromatography afforded the sub-title compound.

### (c) 2-(2-Benzyloxy-5-iodophenyl)-5-methylbenzoxazole

An oven dried ACE pressure tube was charged with 2-(2-benzyloxy-5-bromophenyl)-5-methylbenzoxazole (e.g. 2.51 mmol; see step (b) above), CuI (e.g. 1.26 mmol) and NaI (e.g. 5.04 mmol). The reaction tube was purged with argon and 1,4-dioxane (e.g. 16 mL) was added followed by N,N′-dimethyl-1,2-diaminoethane (e.g. 1.26 mmol). The reaction mixture was heated at 130° C. for 18 h. The mixture was filtered through Celite®. Solvent removal under reduced pressure and chromatography afforded the sub-title compound.

### (d) N-[4-Benzyloxy-3-(5-methylbenzoxazol-2-yl)phenyl]-2-hydroxybenzamide

The title compound was prepared from 2-(2-benzyloxy-5-iodophenyl)-5-methylbenzoxazole (see step (c) above) and 2-trifluoromethylbenzamide in accordance with Example 2(c).

200 MHz ¹H-NMR (DMSO-d₆, ppm) δ 10.68 (1H, s) 8.54 (1H, d, J=2.7 Hz) 7.91-7.57 (9H, m) 7.48-7.30 (4H, m) 7.26 (1H, dd, J=8.6, 1.4 Hz) 5.34 (2H, s) 2.46 (3H, s).

### Example 4

### 2,5-Dichloro-N-{4-chloro-3-[5-(4-isopropoxyphenyl)benzoxazol-2-yl]phenyl}benzenesulfonamide

### (a) 4′-Isopropoxy-3-nitrobiphenyl-4-ol

An oven dried ACE® pressure tube was charged with 4-bromo-2-nitrophenol (654 mg, 3.00 mmol), 4-isopropoxyboronic acid (810 mg, 4.50 mmol), K₂CO₃ (1.66 g, 12.00 mmol) Pd(OAc)₂ (67 mg, 0.3 mmol) and DABCO (67 mg, 0.6 mmol). The reaction tube was purged with argon and acetone (30 mL) was added. The reaction mixture was heated at 110° C. for 48 hours. The mixture was poured in water (100 mL) and extracted with EtOAc. The combined extracts were washed with water and brine and then dried over Na₂SO₄. Concentration under reduced pressure and purification by chromatography afforded the sub-title compound (710 mg, 87%).

### (b) 3-Amino-4′-isopropoxybiphenyl-4-ol

A solution of 4′-isopropoxy-3-nitrobiphenyl-4-ol (710 mg, 2.6 mmol; see step (a) above) in EtOAc (50 mL) and EtOH (50 mL) was hydrogenated in the presence of 10% Pd—C (350 mg) at room temperature for 1 hour. The mixture was filtered through Celite®. Solvent removal under reduced pressure afforded the sub-title compound (600 mg, 95%).

### (c) 2-(2-Chloro-5-nitrophenyl)-5-(4-isopropoxyphenyl)benzoxazole

The sub-title compound was prepared from 3-amino-4′-isopropoxybiphenyl-4-ol (see step (b) above) and 2-chloro-5-nitrobenzoyl chloride in accordance with Example 1 step (a).

### (d) 4-Chloro-3-[5-(4-isopropoxyphenyl)benzoxazol-2-yl]phenylamine

To a stirred suspension of 2-(2-chloro-5-nitrophenyl)-5-(4-isopropoxyphenyl)benzoxazole (e.g. 11.35 mmol; see step (c) above) in EtOH (e.g. 60 mL) was added aq. saturated NH₄Cl (e.g. 25 mL) and Fe powder (e.g. 64.9 mmol). After heating under reflux for 30 min, the reaction was filtered through a celite, EtOAc (300 mL) was added, the organic phase was washed with aq. saturated NaHCO₃, brine and dried over Na₂SO₄. Concentration and purification by chromatography afforded the sub-title compound.

### (e) 2,5-Dichloro-N-{4-chloro-3-[5-(4-isopropoxyphenyl)benzoxazol-2-yl]phenyl}benzenesulfonamide

To a cooled solution of 4-chloro-3-[5-(4-isopropoxyphenyl)-benzoxazol-2-yl]phenylamine (e.g. 1.1 mmol; see step (d) above) in dry pyridine (e.g. 15 mL), 2,5-dichlorobenzenesulfonyl chloride (e.g. 1.31 mmol) was added. After stirring at room temperature for 4 h, the mixture was poured in water (50 mL) and extracted with EtOAc. The combined extracts were washed with water and brine and then dried over Na₂SO₄. Concentration under reduced pressure and purification by chromatography afforded the title compound.

200 MHz ¹H-NMR (DMSO-d₆, ppm) δ 11.32 (1H, s) 8.10 (1H, d, J=2.2 Hz) 8.05 (1H, d, J=1.6 Hz) 7.61 (1H, d, J=2.6 Hz) 7.85 (1H, d, J=8.6 Hz) 7.76 (1H, dd, J=2.2, 8.6 Hz) 7.73-7.58 (5H, m) 7.33 (1H, dd, J=8.8, 2.7 Hz) 7.05-6.97 (2H, m) 4.66 (1H, septet, J=6.0 Hz) 1.28 (6H, d, J=6.0 Hz).

### Example 5

### N-[2-Amino-3′,5′dimethyl-5-(5-methylbenzoxazol-2-yl)-biphenyl-3-yl]-2-trifluoromethylbenzamide

### (a) 5-Methyl-2-(4-nitrophenyl)benzoxazole

The sub-title compound was prepared from 2-amino-4-methylphenol and 4-nitrobenzoyl chloride in accordance with Example 1(a).

### (b) 4-(5-Methylbenzoxazol-2-yl)phenylamine

The sub-title compound was prepared from 5-methyl-2-(4-nitrophenyl)benzoxazole (see step (a) above) in accordance with Example 1(b).

### (c) 2-Bromo-4-(5-methylbenzoxazol-2-yl)phenylamine

To stirred hot (65° C.) solution of 4-(5-methylbenzoxazol-2-yl)phenylamine (1.01 g, 5.0 mmol; see step (b) above) in glacial acetic acid (20 mL) was added dropwise within 30 min a solution of bromine in glacial acetic acid (280 μL, 5.5 mmol) whereupon a yellowish precipitate formed. After stirring at 65° C. for 30 min, an additional solution of bromine (100 μL) in glacial acetic acid (3 mL) was added and the reaction was heated at 65° C. for 1 hour. The reaction mixture was cooled, poured into ice water, the resulting precipitate filtered, washed with water and dried in vacuo. Concentration under reduced pressure and purification by filtration through a silica gel afforded the sub-title compound (1.13 g, 74%).

### (d) N-[2-Bromo-4-(5-methylbenzoxazol-2-yl)phenyl]-2,2-dimethylpropionamide

2,2-Dimethylpropionyl chloride (1.3 mL, 10.6 mmol) was added to a mixture of 2-bromo-4-(5-methylbenzoxazol-2-yl)phenylamine (1.03 g, 3.4 mmol; see step (c) above), triethylamine (2.85 mL, 20.3 mmol) and DMAP (1.04 g, 8.5 mmol) in dry CH₂Cl₂ (50 mL) and the reaction was stirred for 4 days. Additional 2,2-dimethylpropionyl chloride (1.04 mL, 8.5 mmol), triethylamine (2.3 mL, 16.5 mmol) and DMAP (416 mg, 3.4 mmol) were then added and the reaction was refluxed for 10 hours. After dilution with 75 mL CH₂Cl₂ the reaction mixture was washed with water (3×75 mL), brine and dried over Na₂SO₄. Concentration under reduced pressure and purification by chromatography afforded the sub-title compound (815 mg, 62%).

### (e) N-[3-Bromo-3′, 5′-dimethyl-5-(5-methylbenzoxazol-2-yl)biphenyl-2-yl]-2,2,2-trifluoroacetamide

The sub-title compound was prepared from N-[2-bromo-4-(5-methylbenzoxazol-2-yl)-phenyl]-2,2-dimethylpropionamide (see step (d) above) in accordance with Example 2(b).

### (f) 3-Bromo-3′,5′-dimethyl-5-(5-methylbenzoxazol-2-yl)-biphenyl-2-ylamine

A solution of N-[3-bromo-3′,5′-dimethyl-5-(5-methylbenzoxazol-2-yl)-biphenyl-2-yl]-2,2,2-trifluoroacetamide (325 mg, 0.65 mmol; see step (e) above) in methyl alcohol (25 mL) was added to a dry K₂CO₃ (450 mg, 3.25 mmol). After addition of water (1.5 mL) the reaction was stirred overnight and then heated under reflux for 4.5 hours. The reaction mixture was concentrated and the residue was partitioned between CH₂Cl₂ (25 mL) and a mixture of water (25 mL) and 1 M NaOH (3 mL). The basic water layer was additionally extracted with CH₂Cl₂ and the organic extracts were combined, washed with brine and dried over Na₂SO₄. Concentration under reduced pressure and purification by chromatography afforded the sub-title compound (189 mg, 71%).

### (g) N-[2-Amino-3′,5′-dimethyl-5-(5-methylbenzoxazol-2-yl)biphenyl-3-yl]-2-trifluoromethylbenzamide

The title compound was prepared from 3-bromo-3′,5′-dimethyl-5-(5-methylbenzoxazol-2-yl)biphenyl-2-ylamine (see step (f) above) and 2-trifluoromethylbenzamide in accordance with Example 2(c).

200 MHz ¹H-NMR (CDCl₃, ppm) δ 7.97 (1H, s) 7.83-7.77 (1H, m) 7.76-7.61 (4H, m) 7.38-7.35 (1H, m) 7.12 (1H, d, J=8.3 Hz) 7.01 (1H, dd, J=8.3, 1.2 Hz) 6.99-6.95 (2H, m) 6.91-6.86 (1H, m) 6.84 (1H, s) 4.40 (2H, s) 2.43 (3H, s) 2.26 (6H, s).

### Example 6

### N-[3-(5-Methylbenzoxazol-2-yl)-4-((E)-3-phenalallyloxy)phenyl]-2-trifluoro-methylbenzamide

### (a) 2-[5-Bromo-2-((E)-3-phenylallyloxy)phenyl]-5-methylbenzoxazole

The sub-title compound was prepared from 4-bromo-2-(5-methylbenzoxazol-2-yl)phenol (see Example 3(a)) and cinnamyl bromide in accordance with Example 3(b).

### (b) 2-[5-Iodo-2-((E)-3-phenylallyloxy)phenyl]-5-methylbenzoxazole

The sub-title compound was prepared from 2-[5-bromo-2-((E)-3-phenylallyloxy)phenyl]-5-methylbenzoxazole (see step (a) above) in accordance with Example 3(c).

### (c) N-[3-(5-Methylbenzoxazol-2-yl)-4-((E)-3-phenylallyloxy)phenyl]-2-trifluoromethylbenzamide

The title compound was prepared from 3-(5-methylbenzoxazol-2-yl)-4-((E)-3-phenylallyloxy)-N-(2-trifluoromethylphenyl)benzamide (see step (b) above) and 2-trifluoromethylbenzamide in accordance with Example 2(c).

200 MHz ¹H-NMR (DMSO-d₆, ppm) δ 10.68 (1H, s) 8.53 (1H, d, J=2.6 Hz) 7.91-7.62 (7H, m) 7.52-7.45 (2H, m) 7.41-7.22 (5H, m) 6.97 (1H, d, J=16.0) 6.56 (1H, dt, J=16.0, 5.0 Hz) 4.92 (2H, d, J=5.0 Hz) 2.46 (3H, s).

### Example 7

### N-[3-(5-tert-Butylbenzoxazol-2-yl)-5-(3-chlorophenoxymethyl)phenyl]-2-trifluoromethylbenzamide

### (a) 3-Chlorocarbonyl-5-nitrobenzoic Acid methyl ester

A mixture of 5-nitroisophthalic acid monomethyl ester (5.0 g, 22.2 mmol), thionyl chloride (2.5 mL, 33.3 mmol) and a few drops of DMF in CH₂Cl₂ (15 mL) was heated under reflux for 2 h. Solvent removal under reduced pressure and washing with dry hexanes afforded the sub-title compound (5.4 g, 99%).

### (b) 3-(5-tert-Butylbenzoxazol-2-yl)-5-nitrobenzoic Acid methyl ester

The sub-title compound was prepared from 2-amino-4-tert-butylphenol and 3-chlorocarbonyl-5-nitrobenzoic acid methyl ester (see step (a) above) in accordance with Example 1(a)

### (c) 3-(5-tert-Butylbenzoxazol-2-yl)-5-nitrobenzoic Acid

A mixture of 3-(5-tert-butylbenzoxazol-2-yl)-5-nitrobenzoic acid methyl ester (1.5 g, 4.23 mmol; see step (b) above) and an aqueous 2 M NaOH solution (10 mL, 20 mmol) was heated under reflux in dioxane (10 mL) for 1 h. After cooling the reaction was poured into aqueous 1 M HCl (100 mL) and extracted with EtOAc. Solvent removal and recrystallization from EtOAc-petroleum ether afforded the sub-title compound (1.2 g, 83%)

### (d) [3-(5-tert-Butylbenzoxazol-2-yl)-5-nitrophenyl]methanol

To a solution of 3-(5-tert-butylbenzoxazol-2-yl)-5-nitrobenzoic acid (1.8 g, 5.3 mmol; see step (c) above) in dry THF (80 mL) at 0° C. was added a 1 M borane-THF complex (21.2 mL, 21.2 mmol) and the resulting solution was heated in an ACE pressure tube at 100° C. for 72 hours. The reaction mixture was cooled to rt, poured into aqueous 1 M HCl (200 mL) and extracted with EtOAc (3×100 mL). The combined organic extracts were washed with brine and dried over Na₂SO₄. Concentration under reduced pressure and purification by chromatography afforded the sub-title compound (690 mg, 40%).

### (e) 5-tert-Butyl-2-[3-(3-chlorophenoxymethyl)-5-nitrophenyl]benzoxazole

To a cooled (0° C.) solution of [3-(5-tert-butylbenzoxazol-2-yl)-5-nitrophenyl]methanol (230 mg, 0.7 mmol; see step (d) above) and Ph₃P (220 mg, 0.84 mmol) in dry THF (10 mL) was added neat 3-chlorophenol (110 μL, 1.1 mmol), followed by dropwise addition (within 10 min) of diisopropyl azodicarboxylate (220 μL, 1.12 mmol) in THF (5 mL). After stirring for 30 min at ambient temperature, the mixture was concentrated under reduced pressure. Purification by chromatography afforded the sub-title compound (300 mg, 98%).

### (f) 3-(5-tert-Butylbenzoxazol-2-yl)-5-(3-chlorophenoxymethyl)phenylamine

The sub-title compound was prepared from 5-tert-butyl-2-[3-(3-chlorophenoxymethyl)-5-nitrophenyl]benzoxazole (see step (e) above) in accordance with Example 4(d).

### (g) N-[3-(5-tert-Butylbenzoxazol-2-yl)-5-(3-chlorophenoxymethyl)phenyl]-2-trifluoromethylbenzamide

The title compound was prepared from 3-(5-tert-butylbenzoxazol-2-yl)-5-(3-chlorophenoxymethyl)phenylamine (see step (f) above) and 2-trifluoromethylbenzoyl chloride in accordance with Example 1(c).

200 MHz ¹H-NMR (DMSO-d₆, ppm) δ 10.92 (1H, br s) 8.69-8.66 (1H, m) 8.04-7.99(1H, m) 7.91-7.67 (7H, m) 7.49 (1H, dd, J=8.7, 1.9 Hz) 7.38-7.27 (1H, m) 7.16 (1H, dd, J=2.1 Hz) 7.08-6.98 (2H, m) 5.28 (2H, s) 1.34 (9H, s).

### Example 8

### N-[3-(5-tert-Butylbenzoxazol-2-yl)-5-(2-chlorophenoxymethyl)phenyl]-2-trifluoromethylbenzamide

### (a) 5-tert-Butyl-2-[3-(2-chlorophenoxymethyl)-5-nitrophenyl]benzoxazole

The sub-title compound was prepared from [3-(5-tert-butylbenzoxazol-2-yl)-5-nitrophenyl]methanol (see Example 7(d)) and 2-chlorophenol in accordance with Example 7(e).

### (b) 3-(5-tert-Butylbenzoxazol-2-yl)-5-(2-chlorophenoxymethyl)phenylamine

The sub-title compound was prepared from 5-tert-butyl-2-[3-(2-chlorophenoxymethyl)-5-nitrophenyl]benzoxazole (see step (a) above) in accordance with Example 4(d).

### (c) N-[3-(5-tert-Butylbenzoxazol-2-yl)-5-(2-chlorophenoxymethyl)phenyl]-2-trifluoromethylbenzamide

The title compound was prepared from 3-(5-tert-butylbenzoxazol-2-yl)-5-(2-chlorophenoxymethyl)phenylamine (see step (b) above) and 2-trifluoromethylbenzoyl chloride in accordance with Example 1(c).

200 MHz ¹H-NMR (DMSO-d₆, ppm) δ 10.95 (1H, br s) 8.72-8.67 (1H, m) 8.08-8.03 (1H, m) 7.95-7.64 (7H, m) 7.50 (1H, dd, J=8.8, 1.8 Hz) 7.46 (1H, dd, J=7.7, 1.1 Hz) 7.37-7.21 (2H, m) 6.98 (1H, ddd, J=6.8, 6.8, 2.2 Hz) 5.35 (2H, s) 1.35 (9H, s).

### Example 9

### N-[4-Benzyloxy-3-(5-tert-butylbenzoxazol-2-yl)phenyl]-2-trifluoromethylbenzamide

### (a) 4-Bromo-2-(5-tert-butylbenzoxazol-2-yl)phenol

The sub-title compound was prepared from 2-amino-4-tert-butylphenol and 5-bromo-2-hydroxybenzoyl chloride in accordance with Example 3(a).

### (b) 2-(2-Benzyloxy-5-bromophenyl)-5-tert-butylbenzoxazole

The sub-title compound was prepared from 4-bromo-2-(5-tert-butylbenzoxazol-2-yl)phenol (see step (a) above) and chloromethylbenzene in accordance with Example 3(b).

### (c) 2-(2-Benzyloxy-5-iodophenyl)-5-tert-butylbenzoxazole

The sub-title compound was prepared from 2-(2-benzyloxy-5-bromophenyl)-5-tert-butylbenzoxazole (see step (b) above) in accordance with Example 3(c).

### (d) N-[4-Benzyloxy-3-(5-tert-butylbenzoxazol-2-yl)phenyl]-2-trifluoromethylbenzamide

The title compound was prepared from 2-(2-benzyloxy-5-iodophenyl)-5-tertbutylbenzoxazole (see step (c) above) and 2-trifluoromethylbenzamide in accordance with Example 2(c).

200 MHz ¹H-NMR (DMSO-d₆, ppm) δ 10.68 (1H, s) 8.54 (1H, d, J=2.6 Hz) 7.90-7.71 (6H, m) 7.68 (1H, d, J=8.6 Hz) 7.63-7.56 (2H, m) 7.50 (1H, dd, J=8.6, 1.9 Hz) 7.47-7.27 (4H, m) 5.34 (2H, s) 1.37 (9H, s).

### Example 10

### N-[3-(5-tert-Butylbenzoxazol-2-yl)-4-(3-phenylallyloxy)phenyl]-2-trifluoromethylbenzamide

### (a) 2-[5-Bromo-2-((E)-3-phenylallyloxy)phenyl]-5-tert-butylbenzoxazole

The sub-title compound was prepared from 4-bromo-2-(5-tert-butylbenzoxazol-2-yl)phenol (see Example 9(a)) and cinnamyl bromide in accordance with Example 3(b).

### (b) 5-tert-Butyl-2-[5-iodo-2-((E)-3-phenylallyloxy)phenyl]benzoxazole

The sub-title compound was prepared from 2-[5-bromo-2-((E)-3-phenylallyloxy)phenyl]-5-tert-butylbenzoxazole (see step (a) above) in accordance with Example 3(c).

### (c) N-[3-(5-tert-Butylbenzoxazol-2-yl)-4-(3-phenylallyloxy)phenyl]-2-trifluoromethylbenzamide

The title compound was prepared from 5-tertbutyl-2-[5-iodo-2-((E)-3-phenylallyloxy)phenyl]benzoxazole (see step (b) above) and 2-trifluoromethylbenzamide in accordance with Example 2(c).

200 MHz ¹H-NMR (DMSO-d₆, ppm) δ 10.68 (1H, s) 8.54 (1H, d, J=2.6 Hz) 7.90-7.68 (6H, m) 7.70 (1H, d, J=8.6 Hz) 7.54-7.45 (3H, m) 7.41-7.22 (4H, m) 6.99 (1H, d, J=16.1) 6.56 (1H, dt, J=16.1, 4.9 Hz) 4.92 (2H, d, J=4.9 Hz) 1.37 (9H, s).

### Example 11

### N-[3 (5-tert-Butylbenzoxazol-2-yl)-4-(3-(phenylpropoxy)phenyl]-2-trifluoromethylbenzamide

A solution of N-[3-(5-tert-butylbenzoxazol-2-yl)-4-(³-phenylallyloxy)phenyl]-2-trifluoromethylbenzamide (100 mg, 0.18 mmol; see Example 10 step (c)) in EtOAc (20 mL) was hydrogenated in the presence of 10% Pd—C (50 mg) at room temperature for 2 hours. The mixture was filtered through Celite®. Solvent removal under reduced pressure and chromatography afforded the title compound (71 mg, 71%).

200 MHz ¹H-NMR (DMSO-d₆, ppm) δ 10.66 (1H, s) 8.52 (1H, d, J=2.6 Hz) 7.90-7.69 (6H, m) 7.66 (1H, d, J=8.6 Hz) 7.50 (1H, dd, J=8.6, 1.9 Hz) 7.33-7.14 (6H, m) 4.12 (2H, t, J=6.0 Hz) 2.93-2.82 (2H, m) 2.16-1.99 (2H, m) 1.37 (9H, s).

### Example 12

### 2,5-Dichloro-N-{4-chloro-3-[5-(4-isopropoxyphenyl)benzoxazol-2-yl]phenyl}benzamide

The title compound was prepared from 4-chloro-3-[5-(4-isopropoxyphenyl)benzoxazol-2-yl]phenylamine (see Example 4(d)) and 2,5-dichlorobenzoyl chloride in accordance with Example 1(c).

200 MHz ¹H-NMR (DMSO-d₆, ppm) δ 1.01 (1H, s) 8.71 (1H, d, J=2.6 Hz) 8.09 (1H, d, J=1.4 Hz) 7.92-7.82 (3H, m) 7.77-7.59 (6H, m) 7.07-6.98 (2H, m) 4.68 (1H, septet, J=6.0 Hz) 1.30 (6H, d, J=6.0 Hz).

### Example 13

### 2,5-Dichloro-N-{3-[5-(4-isopropoxyphenyl)benzoxazol-2-yl]phenyl}benzenesulfonamide

### (a) 3-[5-(4-Isopropoxyphenyl)benzoxazol-2-yl]phenylamine

The sub-title compound was prepared by hydrogenation of 2-(2-chloro-5-nitrophenyl)-5-(4-isopropoxyphenyl)benzoxazole (see Example 4(c)), which hydrogenation was performed in the presence of solvent (e.g. EtOAc, EtOH or a mixture thereof) and 10% Pd—C, which reaction mixture was stirred at about room temperature for about 4 hours. The resulting mixture may be filtered through Celite®, after which solvent removal and chromatography may afford the sub-title compound.

### (b) 2,5-Dichloro-N-{3-[5-(4-isopropoxyphenyl)benzoxazol-2-yl]phenyl}benzenesulfonamide

The title compound was prepared from 3-[5-(4-isopropoxyphenyl)benzoxazol-2-yl]phenylamine (see step (a) above) and 2,5-dichlorobenzenesulfonyl chloride in accordance with Example 4(e).

200 MHz ¹H-NMR (DMSO-d₆, ppm) δ 11.21 (1H, s) 8.09 (1H, d, J=2.2 Hz) 8.02-7.97 (2H, m) 7.91-7.79 (2H, m) 7.75 (1H, dd, J=8.6, 2.2 Hz) 7.74-7.60 (4H, m) 7.52 (1H, dd, J=8.1, 8.1 Hz) 7.38 (1H, ddd, J=8.1, 2.2, 1.0 Hz) 7.06-6.97 (2H, m) 4.66 (1H, septet, J=6.0 Hz) 1.29 (6H, d, J=6.0 Hz).

### Example 14

### 3-(5-tert-Butylbenzoxazol-2-yl)-N-(3-chloro-2-methylphenyl)-N-methyl-5-(2-trifluoromethylbenzoylamino)benzamide

### (a) 3-Chlorocarbonyl-5-nitrobenzoic Acid methyl ester

A mixture of 5-nitroisophthalic acid monomethyl ester (5.0 g, 22.2 mmol), thionyl chloride (2.5 mL, 33.3 mmol) and a few drops of DMF in CH₂Cl₂ (15 mL) was heated under reflux for 2 h. Solvent removal under reduced pressure and washing with dry hexanes afforded the sub-title compound (5.4 g, 99%).

### (b) 3-(5-tert-Butylbenzoxazol-2-yl)-5-nitrobenzoic Acid methyl ester

The sub-title compound was prepared from 2-amino-4-tert-butylphenol and 3-chlorocarbonyl-5-nitrobenzoic acid methyl ester (see step (a) above) in accordance with Example 1(a).

### (c) 3-(5-tert-Butyl-benzoxazol-2-yl)-5-nitrobenzoic Acid

A mixture of 3-(5-tert-butylbenzoxazol-2-yl)-5-nitrobenzoic acid methyl ester (1.5 g, 4.23 mmol; see step (b) above) and an aqueous 2 M NaOH solution (10 mL, 20 mmol) was heated under reflux in dioxane (10 mL) for 1 h. After cooling the reaction was poured into aqueous 1 M HCl (100 mL) and extracted with EtOAc. Solvent removal and recrystallization from EtOAc-petroleum ether afforded the sub-title compound (1.2 g, 83%)

### (d) 3-(5-tert-Butylbenzoxazol-2-yl)-5-nitrobenzoyl chloride

The sub-title compound was prepared from 3-(5-tert-butylbenzoxazol-2-yl)-5-nitrobenzoic acid (see step (c) above) in accordance with Example 7(a).

### (e) 3-(5-tert-Butylbenzoxazol-2-yl)-N-(3-chloro-2-methylphenyl)-5-nitrobenzamide

A mixture of 3-(5-tert-butylbenzoxazol-2-yl)-5-nitrobenzoyl chloride (1.46 g, 4.1 mmol), 3-chloro-2-methylphenylamine (585 μL, 4.9 mmol), triethylamine (1.73 mL, 12.3 mmol) and 4-dimethylaminopyridine (125 mg, 1.03 mmol) in dry acetonitrile (50 mL) was stirred overnight at ambient temperature. The reaction was poured into water and extracted with EtOAc. The combined organic extracts were washed with aqueous 1 M HCl and then with aqueous saturated NaHCO₃. Concentration and purification by chromatography afforded the title compound (1.07 g, 56%).

### (f) 3-(5-tert-Butylbenzoxazol-2-yl)-N-(3-chloro-2-methylphenyl)-N-methyl-5-nitrobenzamide

The sub-title compound was prepared from 3-(5-tert-butylbenzoxazol-2-yl)-N-(3-chloro-2-methylphenyl)-5-nitrobenzamide (see step (e) above) and methyl iodide. For example, dry DMF may be added gradually via cannula to a suspension of 60% NaH (e.g. washed twice with dry Et₂O prior to use) in DMF at 0° C. After warming to room temperature, the reaction may be stirred for 30 min, whereupon neat MeI may be added. The reaction mixture may be stirred at room temperature for a further 24 h, after which the mixture may be poured into aq. 1N HCl and extracted with EtOAc. Combined extracts may be washed with water and brine and then dried over Na₂SO₄. Concentration and purification by chromatography may then afford the sub-title compound.

### (g) 3-Amino-5-(5-tert-butylbenzoxazol-2-yl)-N-(3-chloro-2-methylphenyl)-N-methylbenzamide

The sub-title compound was prepared from 3-(5-tert-butylbenzoxazol-2-yl)-N-(3-chloro-2-methylphenyl)-N-methyl-5-nitrobenzamide (see step (f) above) in accordance with Example 4(d).

### (h) 3-(5-tert-Butylbenzoxazol-2-yl)-N-(3-chloro-2-methylphenyl)-N-methyl-5-(2-trifluoromethylbenzoylamino)benzamide

The title compound was prepared in accordance with Example 1(c) from 3-amino-5-(5-tert-butylbenzoxazol-2-yl)-N-(3-chloro-2-methylphenyl)-N-methyl-benzamide (see step (g) above) and 2-trifluoromethylbenzoyl chloride.

200 MHz ¹H-NMR for major amide rotamer (DMSO-d₆, ppm) δ 10.8 (1H, s) 8.47-8.45 (1H, m) 7.87-7.64 (8H, m) 7.49 (1H, dd, J=8.8, 1.8 Hz) 7.31-7.19 (2H, m) 7.10 (1H, dd, J=8.0, 8.0 Hz) 3.28 (3H, s) 2.33 (3H, s) 1.34 (9H, s).

### Example 15

Title compounds of the examples were tested in the biological test described above and were found to exhibit 50% inhibition of mPGES-1 at a concentration of 10 μM or below. For example, the following representative compounds of the examples exhibited the following IC₅₀ values:

Example 4: 600 nM

Example 5: 130 nM

Example 7: 270 nM

1. A compound of formula I, 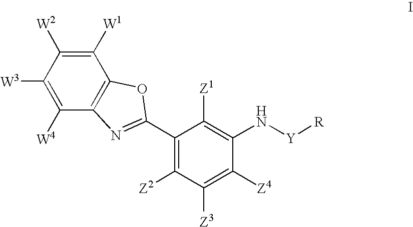
wherein
R represents aryl or heteroaryl, both of which are optionally substituted by one or more substituents selected from X¹;
Y represents —C(O)— or —S(O)₂—;
W¹ to W⁴ independently represent hydrogen or a substituent selected from X²;
Z¹ to Z⁴ independently represent hydrogen or a substituent selected from X³;
X¹, X² and X³ independently represent halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ;
R^{3b} to R^{3h}, R^{3j}, R^{3k}, R³ⁿ, R^{4a} to R⁴ⁱ, R^{5a}, R^{5b}, R^{5d} and R^{5f} to R⁵ⁱ independently represent H or R^{3a}; or
any of the pairs R^{4a} and R^{5a}, R^{4b} and R^{5b}, R^{4d} and R^{5d}, R^{4f} and R^{5f}, R^{4g} and R^{5g}, R^{4h} and R^{5h} or R⁴ⁱ and R⁵ⁱ may be linked together to form a 3- to 6-membered ring, which ring optionally contains a further heteroatom in addition to the nitrogen atom to which these substituents are necessarily attached, and which ring is optionally substituted by F, Cl, ═O or R^{3a};
R³ⁱ, R^{3m} and R^{3p} independently represent R^{3a};
R^{3a} represents aryl, heteroaryl (which latter two groups are optionally substituted by one or more substituents selected from G³) or C₁₋₆ alkyl optionally substituted by one or more substituents selected from aryl, heteroaryl (which latter two groups are optionally substituted by one or more substituents selected from G⁴), F, Cl, ═O, —OR^{6a} and —N(R^{6b})R^{7b};
R^{6a} and R^{6b} independently represent H, aryl, heteraryl (which latter two groups are optionally substituted by one or more groups selected from G⁵) or C₁₋₆ alkyl optionally substituted by one or more substituents selected from aryl, heteroaryl (which latter two groups are optionally substituted by one or more groups selected from G⁶), F, Cl, ═O, —OR^{8a}, —N(R^{9a})R^{10a} and —S(O)₂-G¹;
R^{7b} represents H, —S(O)₂CH₃, —S(O)₂CF₃ or C₁₋₆ alkyl optionally substituted by one or more substituents selected from F, Cl, ═O, OR^{11a}, —N(R^{12a})R^{13a} and —S(O)₂-G²; or R^{6b} and R^{7b} may be linked together to form a 3- to 6-membered ring, which ring optionally contains a further heteroatom in addition to the nitrogen atom to which these substituents are necessarily attached, and which ring is optionally substituted by F, Cl, ═O or C₁₋₃ alkyl optionally substituted by one or more fluoro atoms;
G¹ and G² independently represent —N(R^{14a})R^{15a} or C₁₋₆ alkyl optionally substituted by one or more substituents selected from F, Cl, ═O, —OR^{16a} and —N(R^{17a})R^{18a};
R^{8a} and R^{11a} independently represent H, —CH₃, —CH₂CH₃ or —CF₃;
R^{9a}, R^{10a}, R^{12a}, R^{13a}, R^{14a}, R^{15a}, R^{16a}, R^{17a} and R^{18a} independently represent H, —CH₃ or —CH₂CH₃;
G³, G⁴, G⁵ and G⁶ independently represent halo, —R^{20a}, —CN, —C(O)R^{20b}, —C(O)OR^{20c}, —C(O)N(R^{21a})R^{22a}, —N(R^{21b})R^{22b}, —N(R^{20d})C(O)R^{21c}, —N(R^{20e})C(O)N(R^{21d})R^{22d}, —N(R^{20f})C(O)OR^{21e}, —N₃, —NO₂, —N(R^{20g})S(O)₂N(R^{21f})R^{22f}, —OR^{20h}, —OC(O)N(R^{21g})R^{22g}, —OS(O)₂R²⁰ⁱ, —S(O)ₘR^{20j}, —N(R^{20k})S(O)₂R^{20m}, —OC(O)R²⁰ⁿ, —OC(O)OR^{20p}, —S(O)₂N(R^{21h})R^{22h} or —OS(O)₂N(R²¹ⁱ)E²²ⁱ;
m represents 0, 1 or 2;
R^{20b} to R^{20h}, R^{20j}, R^{20k}, R²⁰ⁿ, R^{21a} to R²¹ⁱ, R^{22a}, R^{22b}, R^{22d} and R^{22f} to R²²ⁱ independently represent H or R^{20a}; or
any of the pairs R^{21a} and R^{22a}, R^{21b} and R^{22b}, R^{21d} and R^{22d}, R^{21f} and R^{22f}, R^{21g} and R^{22g}, R^{21h} and R^{22h} or R²¹ⁱ and R^{22l} may be linked together to form a 3- to 6-membered ring, which ring optionally contains a further heteroatom in addition to the nitrogen atom to which these substituents are necessarily attached, and which ring is optionally substituted by F, Cl, ═O or R^{20a};
R²⁰ⁱ, R^{20m} and R^{20p} independently represent R^{20a};
R^{20a} represents C₁₋₆ alkyl (optionally substituted by one or more substituents selected from ═O and T¹) or aryl or heteroaryl (which latter two groups are optionally substituted by one or more substituents selected from T²);
T¹ and T² independently represent F, Cl, —OR^{23a} or —N(R^{23b})R^{24b};
R^{23a}, R^{23b} and R^{24b} independently represent H, C₁₋₃ alkyl (optionally substituted by one or more substituents selected from ═O and T³) or aryl or heteroaryl (which latter two groups are optionally substituted by one or more substituents selected from T⁴);
T³ and T⁴ independently represent F, Cl, —OR^{25a} or —N(R^{25b})R^{26b};
R^{25a}, R^{25b} and R^{26b} independently represent H or C₁₋₃ alkyl optionally substituted by one or more fluoro atoms;
wherein:
at least one X¹, X² or X³ group is present and represents —R^{3a}, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} or —OS(O)₂N(R⁴ⁱ)R⁵ⁱ, in which the foregoing groups contain at least one (e.g. one) aryl or heteroaryl group (both of which are optionally substituted as defined above),
or a pharmaceutically acceptable salt thereof,
provided that:
when Y represents —C(O)—:
(a) W¹ to W⁴ and Z¹ to Z⁴ all represent H, then R does not represent 5-trifluoromethyl-N-(4-chlorophenyl)-pyrazol-4-yl;
(b) W¹ to W⁴, Z¹, Z² and Z⁴ all represent H, R represents unsubstituted phenyl and Z³ represents —N(R^{d})C(O)R^{4c} in which R^{3d} represents H, then R^{4c} does not represent unsubstituted phenyl;
(c) W¹, W³, W⁴ and Z¹ to Z⁴ represent H, R represents unsubstituted phenyl, then W² does not represent 2-furanyl or 2-fluorophenyl;
(d) W¹ to W⁴, Z¹ and Z⁴ all represent H, Z2 represents —OH and Z³ represents —C(O)R^{3b}, then R and R^{3b} do not both represent unsubstituted phenyl or 4-fluorophenyl;
(e) W¹, W⁴, Z¹ and Z³ all represent hydrogen:
(I) W² and Z² represent hydrogen, Z⁴ represents chloro, then when:
(A) W³ represents —CH(CH₂CH₃)CH₃ (i.e. 1-methylpropyl), then R does not represent 4-(benzyloxy)-phenyl, 3-(benzyloxy)phenyl or 4-(phenyl)phenyl;
(B) W³ represents isopropyl, then R does not represent 3-(benzyloxy)phenyl;
(II) W² and Z⁴ represent hydrogen, W³ represents ethyl:
(A) Z² represents hydroxy, then R does not represent (4-phenyl)phenyl;
(B) Z² represent hydrogen, then R does not represent 3-(2-oxo-2H-1-benzopyran-3-yl)-phenyl (i.e. 3-(2-oxo-2H-chromen-3-yl)-phenyl);
(III) W³ and Z² represent hydrogen, W² represents methyl, Z⁴ represents chloro, then R does not represent 3-(benzyloxy)phenyl;
(IV) W², W³, Z² and Z⁴ represent hydrogen, then R does not represent 3-phenoxymethyl)phenyl or 2-(2,4-dimethylphenyl)-2,3-dihydro-1,3-dioxo-1H-isoindol-5-yl;
(f) W⁴ and Z³ represent hydrogen, R represents 2-furanyl substituted in the 5-position (only) by X¹, then:
(I) when W¹, W², Z¹ and Z² represent hydrogen:
(A) Z⁴ represents hydrogen, W³ represents 1-methylpropyl, then X¹ does not represent 3-nitrophenyl;
(B) Z⁴ represents hydrogen, W³ represents isopropyl, then X¹ does not represent 2,5-dichlorophenyl;
(C) Z⁴ represents hydrogen, W³ represents chloro, then X¹ does not represent 2,3-dichlorophenyl;
(D) Z⁴ represents methyl, W³ represents isopropyl, then X¹ does not represent 3-chloro-4-methylphenyl;
(II) when W¹ represents hydrogen, W² and W³ represent methyl:
(A) Z¹ and Z² represent hydrogen, Z⁴ represents —OCH₃, then X¹ does not represent 2,5-dichlorophenyl;
(B) Z¹ represents methyl, Z² and Z⁴ represent hydrogen, then X¹ does not represent 4-(carboethoxy)phenyl;
(C) Z¹, Z² and Z⁴ represent hydrogen, then X¹ does not represent 2,5-dichlorophenyl;
(III) W¹, W², W³ and Z² represent hydrogen:
(A) Z⁴ represents hydrogen, Z¹ represents methyl, then X¹ does not represent 3-chloro-4-methylphenyl or 4-bromophenyl;
(B) Z¹ represents hydrogen, Z⁴ represents —OCH₃, then X¹ does not represent 3-chloro-2-methylphenyl;
(IV) X¹ does not represent 2-nitrophenyl when:
(A) W¹, W², Z¹ and Z² represent hydrogen, W³ represents —OCH₃ and Z⁴ represents methyl;
(B) W¹, W², W³, Z¹ and Z² represent hydrogen, and Z⁴ represents —OCH₃;
(C) W¹, W², Z² and Z⁴ represent hydrogen, W³ represents chloro and Z¹ represents methyl;
(D) W¹, Z² and Z⁴ represent hydrogen and Z¹, W² and W³ represent methyl;
(E) W¹, W², Z¹ and Z² represent hydrogen, W³ represents methyl and Z⁴ represents chloro;
(V) X¹ does not represent 4-chlorophenyl when:
(A) W¹ and W³ represent methyl, W² represents hydrogen, and either: Z¹ and Z⁴ represent hydrogen and Z² represents chloro; Z¹ and Z² represent hydrogen and Z⁴ represents methyl; or Z² and Z⁴ represent hydrogen and Z¹ represents methyl;
(B) W¹, W², Z¹ and Z⁴ represent hydrogen, and either: W³ represents ethyl and Z² represents chloro; or W³ represents methyl and Z² represents hydrogen;
(C) W¹, W², Z¹ and Z² represent hydrogen, W³ represents isopropyl and Z⁴ represents methyl;
(VI) X¹ does not represent 3-nitrophenyl when W¹ and W³ represent methyl, W², Z¹ and Z⁴ represent hydrogen, and Z² represents chloro;
(g) W¹, W⁴, Z¹, Z² and Z³ all represent hydrogen, W² and W³ represent methyl, Z⁴ represents —OCH₃, then R does not represent 3-(methoxy)-4-(4-chlorobenzyloxy)-phenyl; and
(h) W¹, W³, W⁴, Z¹, Z², Z³ and Z⁴ represent hydrogen, W² represents X² in which X² represents —N(R^{3d})C(O)R^{4c}, R^{3d} represents hydrogen, then R and R^{3d} do not both represent 3-chlorophenyl, 4-methylphenyl, 4-chlorophenyl or unsubstituted phenyl.

2. A compound as claimed in claim 1, further provided that:
(i) when Y represents —S(O)₂—, W¹, W², W³, W⁴, Z¹, Z² and Z³ represent hydrogen, R represents 4-methylphenyl, then Z⁴ does not represent 2-benzoxazolyl.

3. A compound as claimed in claim 1 or claim 2, wherein W¹ and W⁴ independently represent H.

4. A compound as claimed in claim 1, wherein W² and W³ independently represent X² or H.

5. A compound as claimed in claim 1, wherein Z¹ represents H.

6. A compound as claimed in claim 1, wherein Z², Z³ and Z⁴ independently represent X³ or H.

7. A compound as claimed in claim 1, wherein only one X¹, X² or X³ group is present in which it contains an R^{3a} group containing the essential aryl or heteroaryl group, and when other X¹, X² or X³ groups are present, then they do not represent a group that contains an R^{3a} group containing the essential aryl or heteroaryl group.

8. A compound as claimed in claim 1, wherein X¹, X² or X³ independently represent halo, R^{3a}, —C(O)N(R^{4a})R^{5a}, —N(R^{4b})R^{5b} —N(R^{3d})C(O)R^{4c} or —OR^{3h}.

9. A compound as claimed in claim 7 or claim 8, wherein the X¹, X² or X³ group containing an R^{3a} group containing the essential aryl or heteroaryl group is X² or X³.

10. A compound as claimed in claim 9, wherein the X¹, X² or X³ group is X³.

11. A compound as claimed in claim 1, wherein R^{4a} represents H or an R^{3a} group that does not contain the essential aryl or heteroaryl group.

12. A compound as claimed in claim 1, wherein R^{5a} represents R^{3a}.

13. A compound as claimed in claim 1, wherein R^{4b} and R^{5b} independently represent hydrogen.

14. A compound as claimed in claim 1, wherein R^{3d} represents H.

15. A compound as claimed in claim 1, wherein R^{4c} represents R^{3a}.

16. A compound as claimed in claim 1, wherein R^{3h} represents R^{3a}.

17. A compound as claimed in claim 1, wherein R^{3a} represents aryl (optionally substituted by one substituent selected from G³), C₁₋₄ alkyl optionally substituted by one or more fluoro atoms, or phenyl or —OR^{6a} groups.

18. A compound as claimed in claim 1, wherein the R^{3a} group containing the essential aryl or heteroaryl group represents C₁₋₆ alkyl substituted by one or more substituents selected from aryl, heteroaryl (which latter two groups are optionally substituted by one or more substituents selected from G⁴), —N(R^{6b})R^{7b} and —OR^{6a}, in which R^{6a} and R^{7b} represent aryl, heteroaryl (which latter two groups are optionally substituted by one or more substituents selected from G⁵) or C₁₋₆ alkyl optionally substituted by one or more substituents selected from aryl and heteroaryl (which latter two groups are optionally substituted by one or more substituents selected from G⁶).

19. A compound as claimed in claim 1, wherein R^{6a} represents phenyl optionally substituted by G⁵.

20. A compound as claimed in any one of the preceding claims claim 1, wherein G³ represents halo, R^{20a} or —OR^{20h}.

21. A compound as claimed in claim 1, wherein R^{20h} represents R^{20a}.

22. A compound as claimed in claim 1, wherein R^{20a} represents C₁₋₃ alkyl.

23. A compound as claimed in claim 1, wherein G⁴, G⁵ and G⁶ independently represent halo.

24. A compound as claimed in claim 1, wherein R represents optionally substituted phenyl, naphthyl, pyrrolyl, furanyl, thienyl, imidazolyl, oxazolyl, isoxazolyl, thiazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl or benzodioxanyl.

25. A compound as claimed in claim 1, wherein R represents phenyl optionally substituted by one or two X¹ substituents.

26. A compound as defined in claim 1 but without provisos (b), (d) to (h) and (i) (if applicable), or a pharmaceutically-acceptable salt thereof, for use as a pharmaceutical.

27. A pharmaceutical formulation including a compound as defined in claim 1 but without provisos (b), (d) to (h) and (i) (if applicable), or a pharmaceutically-acceptable salt thereof, in admixture with a pharmaceutically acceptable adjuvant, diluent or carrier.

28. A compound as defined in claim 1 but without the provisos, or a pharmaceutically-acceptable salt thereof, for use in the treatment of a disease in which inhibition of the activity of a member of the MAPEG family is desired and/or required.

29. (canceled)

30. A compound as claimed in claim 28, wherein the member of the MAPEG family is microsomal prostaglandin E synthase-1, leukotriene C₄ synthase and/or 5-lipoxygenase-activating protein.

31. A compound as claimed in claim 30, wherein the member of the MAPEG family is microsomal prostaglandin E synthase-1.

32. A compound as claimed in claim 28 (as appropriate), wherein the disease is inflammation.

33. The method which comprises administering a compound as defined in claim 1 but without the provisos, or a pharmaceutically-acceptable salt thereof, for the treatment of asthma, chronic obstructive pulmonary disease, pulmonary fibrosis, inflammatory bowel disease, irritable bowel syndrome, pain, inflammatory pain, fever, migraine, headache, low back pain, fibromyalgia, a myofascial disorder, a viral infection, a bacterial infection, a fungal infection, dysmenorrhea, a burn, a surgical or dental procedure, a malignancy, hyperprostaglandin E syndrome, classic Bartter syndrome, atherosclerosis, gout, arthritis, osteoarthritis, juvenile arthritis, rheumatoid arthritis, rheumatic fever, ankylosing spondylitis, Hodgkin's disease, systemic lupus erythematosus, vasculitis, pancreatitis, nephritis, bursitis, conjunctivitis, iritis, scleritis, uveitis, wound healing, dermatitis, eczema, psoriasis, stroke, diabetes mellitus, a neurodegenerative disorder, an autoimmune disease, an allergic disorder, rhinitis, an ulcer, coronary heart disease, sarcoidosis, any other disease with an inflammatory component, osteoporosis, osteoarthritis, Paget's disease or a periodontal disease.

34. (canceled)

35. A method of treatment of a disease in which inhibition of the activity of a member of the MAPEG family is desired and/or required, which method comprises administration of a therapeutically effective amount of a compound as defined in claim 1 but without the provisos, or a pharmaceutically-acceptable salt thereof, to a patient suffering from, or susceptible to, such a condition.

36. A method as claimed in claim 35, wherein the member of the MAPEG family is microsomal prostaglandin E synthase-1, leukotriene C₄ synthase and/or 5-lipoxygenase-activating protein.

37. A method as claimed in claim 36, wherein the member of the MAPEG family is microsomal prostaglandin E synthase-1.

38. A combination product comprising:
(A) a compound of formula I, as defined in claim 1 but without the provisos, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

39. (canceled)

40. A combination product which comprises a kit comprising:
(a) a pharmaceutical formulation including a compound of formula I as defined in claim 1 but without the provisos, or a pharmaceutically-acceptable salt thereof in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

41. A process for the preparation of a compound of formula I as defined in claim 1, which comprises:
(i) reaction of a compound of formula II, 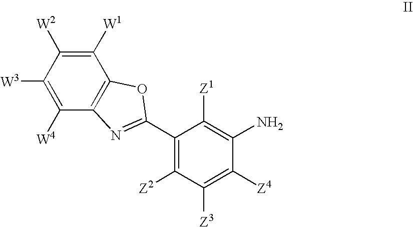
wherein W¹ to W⁴ and Z¹ to Z⁴ are as defined in claim 1, with a compound of formula III,
R—Y—OH III
wherein R and Y are as defined in claim 1; or
(ii) reaction of a compound of formula IV, 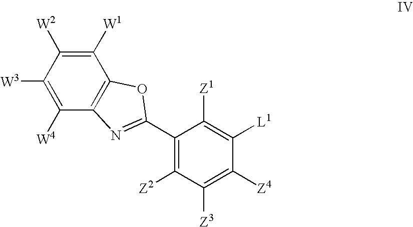
wherein L¹ represents a suitable leaving group, and W¹ to W⁴ and Z¹ to Z⁴ are as defined in claim 1, with a compound of formula V,
H₂N—Y—R V
wherein R and Y are as defined in claim 1.

42. A process for the preparation of a pharmaceutical formulation as defined in claim 27, which process comprises bringing into association said compound of formula I but without provisos (b), (d) to (h) and (i) (if applicable), or a pharmaceutically acceptable salt thereof with a pharmaceutically-acceptable adjuvant, diluent or carrier.

43. A process for the preparation of a combination product as defined in claim 38, which process comprises bringing into association said compound of formula I, but without the provisos, or a pharmaceutically acceptable salt thereof with another therapeutic agent that is useful in the treatment of inflammation, and a pharmaceutically-acceptable adjuvant, diluent or carrier.
